SEQUENCE LISTING (2 sequences)

SEQ ID NO: 1 - RNA, 21 nt, Artificial Sequence
  siRNA
aacaagaccuucgacucuucc

SEQ ID NO: 2 - RNA, 21 nt, Artificial Sequence
  siRNA
gcucacagcucaauccuaauc

(19) United States
(12) Patent Application Publication
(10) Pub. No.: US 2023/0183695 A1
(43) Pub. Date: Jun. 15, 2023

(54) COMPOSITIONS AND METHODS FOR THE DELIVERY OF AGENTS TO BIOLOGICAL TARGETS

(71) Applicants: Formulytica Pty Ltd., Mulgrave, Victoria (AU); Singapore Health Services Pte Ltd., Singapore (SG)

(72) Inventors: Richard Buchta, Mulgrave, Victoria (AU); Michael Andrews Luke, Mulgrave, Victoria (AU); Tina Tzee Ling Wong, Singapore (SG); Li Fong Seet, Singapore (SG)

(21) Appl. No.: 17/924,449

(22) PCT Filed: May 21, 2021
(86) PCT No.: PCT/SG2021/050277

§ 371 Date: Nov. 10, 2022

(30) Foreign Application Priority Data
May 22, 2020 (SG) 10202004832R

(51) Int. Cl. C12N 15/113; A61K 47/61; A61K 47/52; A61P 27/00; A61K 47/64

(52) U.S. Cl. CPC C12N 15/113; A61K 47/61; A61K 47/52; A61P 27/00; A61K 47/6455; C12N 2310/14

(57) ABSTRACT

The present invention relates to compositions comprising an anionic polymer, such as hyaluronate or alginate, a cation such as calcium, an anion such as phosphate, and nucleic acids such as siRNA for the SPARC gene. The compositions are used for delivery of nucleic acids to cells to thereby regulate gene expression and treat diseases such as ocular fibrosis disorders.

### INCORPORATION BY CROSS-REFERENCE

The present invention claims priority from Singapore provisional patent application number 10202004832R, the entire content of which is incorporated herein by cross-reference.

### TECHNICAL FIELD

The present invention relates generally to the fields of biology and medicine, and more specifically to compositions and methods for the delivery of agents to biological targets. In certain forms, the present invention provides compositions and methods for delivering therapeutic molecules to target cells and tissues, along with methods for the preparing these compositions.

### BACKGROUND

The identification and/or development of new molecules for drugs is time consuming and expensive. A common impediment to the success of new therapeutics is an efficient means of delivery. Targeted therapies, i.e., therapies which act on specific molecular targets, afford numerous benefits including reduced adverse effects on unaffected tissues and increased effectiveness in achieving therapeutic goals, but are particularly reliant on an effective delivery mode.

Key factors for the successful delivery of small molecules/drugs to specific biological targets include the ability to deliver the small molecule/drug in sufficient quantities for therapeutic efficacy, the ability to deliver the agents over a prolonged period, low toxicity and/or immunogenicity of the delivery vehicle and the provision of protection for the small molecule/drug. Therapeutics based on nucleic acids, e.g., DNA, RNA and locked nucleic acid (LNA) generally require a high degree of protection due to their susceptibility to degradation by nucleases. The provision of a drug over a sustained period via a delivery vehicle frequently suffers from vehicle-associated toxicity and/or the induction of an immune response to the vehicle.

Small interfering RNA (siRNA), also known as “short interfering RNA” and “silencing RNA” is an example of a small molecule with tremendous therapeutic potential due to its ability to substantially silence the expression of a specific gene. siRNA formulations have been developed to treat of a variety of specific disorders including respiratory syncytial virus (RSV) infection and liver diseases. Various modes of siRNA delivery have been trialled including nanoparticles, microparticles, liposomes, exosomes, gels and emulsions, but none have been able to deliver large quantities of siRNA for prolonged periods without adverse effects such as toxicity and/or immunogenicity. Naked siRNA has a short serum half-life due to renal filtration and because of toxicity associated with activation of the innate immune response, including toll-like receptors (TLRs) and cytoplasmic receptors that recognise patterns in short double stranded DNA and RNA.

One example of a disorder for which siRNA shows enormous therapeutic potential is glaucoma, which is the leading cause of irreversible blindness worldwide. Glaucoma is a progressive disease affecting the optic nerve and leading to blindness, and is mainly caused by high intraocular pressure (TOP). Glaucoma filtration surgery (GFS) is the most effective method to lower the TOP and slow disease progression. The aim of glaucoma filtration surgery is to lower the TOP by way of creating a new surgical pathway for aqueous outflow. After a period of time (months) following surgery, scar tissue forms to cover the surgically created pathway, thereby blocking aqueous outflow and leading to elevation of TOP. This post-operative wound healing response is known as subconjunctival fibrosis and is the main obstacle to achieving long-term surgical success. Current standard anti-scarring treatments used with surgery (Mitomycin-C and 5-Fluorouracil) suffer from irreversible blinding complications. An siRNA which targets the expression of the Sparc gene (secreted protein acidic and rich in cysteine) has potential for the prevention and/or treatment of post-GFS fibrosis by modulating collagen production.

As with many other drugs based on small molecules, the success of this siRNA as a therapeutic will depend on the design of an effective mode of delivery.

A need exists for compositions which can safely deliver small molecules to cells and/or into cells in therapeutically effective quantities over a sustained period.

### SUMMARY OF THE INVENTION

The present invention addresses at least one of the problems associated with current compositions and/or methods for the delivery of agents to biological targets.

The present inventors have surprisingly found that using cations to non-covalently complex an anionic polymer to nucleic acids to be delivered to biological targets alleviates problems associated with toxicity and/or immunogenicity of compositions for the delivery of nucleic acids, problems associated with degradation of the nucleic acids, and/or problems associated with poor efficiency of delivery to target cells. The compositions provided herein comprise non-toxic, biocompatible materials which use natural cellular processes to facilitate the delivery of nucleic acids to biological targets.

The present invention relates at least in part to the following embodiments.
Embodiment 1. A composition for the delivery of nucleic acids to cells, the composition comprising:
an anionic polymer,
cations, wherein the cations do not form part of chitosan or protamine, and
the nucleic acids for delivery to the cells,
wherein the anionic polymer, cations and nucleic acids are bonded by noncovalent interactions.
Embodiment 2. The composition according to embodiment 1, wherein the composition comprises anions.
Embodiment 3. The composition according to embodiment 1 or embodiment 2, wherein the anions are selected from the group consisting of: phosphate, monohydrogen phosphate, carbonate, hydrogen carbonate, citrate, sulphate, malate, tartrate, gluconate, aspartate, glutamate, oxalate, malonate, succinate, glutarate, adipate, and any combination thereof.
Embodiment 4. The composition according to any one of embodiments 1 to 3, wherein the composition comprises sodium citrate, ethylenediaminetetraacetic acid (EDTA), malate, tartrate, glutamate, histidine, gluconate, lysine, glutamine, methionine, threonine, or any combination thereof.
Embodiment 5. The composition according to any one of embodiments 1 to 4, wherein the cations are selected from the group consisting of: multivalent metal ions, calcium, magnesium, manganese, iron, zinc, scandium, titanium, vanadium, chromium, cobalt, nickel, copper, or of the following: glutamine, lysine, arginine, polyglutamine, polylysine, polyarginine, ternary amine-containing compounds, quaternary amine-containing compounds, and any combination thereof.
Embodiment 6. The composition according to any one of embodiments 1 to 5, wherein the cations are selected from the group consisting of: calcium, magnesium, polyarginine, and any combination thereof.
Embodiment 7. The composition according to any one of embodiments 1 to 6, wherein the anionic polymer comprises a naturally-occurring anionic polymer.
Embodiment 8. The composition according to any one of embodiments 1 to 7, wherein the anionic polymer is selected from the group consisting of: hyaluronate, pectin, cellulose sulphate, alginate, polyacrylic acid, carboxymethyl cellulose, carboxymethyl, dextran, and any combination thereof.
Embodiment 9. The composition according to any one of embodiments 1 to 8, wherein the anionic polymer comprises a polymer having a molecular weight of between 30 and 300 kDa, between 35 and 250 kDa, between 40 and 200 kDa, between 45 and 150 kDa, between 50 and 120 kDa, between 60 and 100 kDa, between 50 and 90 kDa, between 70 and 90 kDa or between 30 and 100 kDa.
Embodiment 10. The composition according to any one of embodiments 1 to 9, wherein the cations are components of an ionic salt included in the composition.
Embodiment 11. The composition according to any one of embodiments 1 to 10, wherein the noncovalent interactions are generated by the cations.
Embodiment 12. The composition according to any one of embodiments 1 to 11, wherein the nucleic acids for delivery to cells comprise any one or more of: DNA, RNA and locked nucleic acid (LNA).
Embodiment 13. The composition according to embodiment 12, wherein the RNA is selected from the group consisting of: siRNA, miRNA, mRNA, RNA aptamers, ribozymes, circular RNA, and any combination thereof.
Embodiment 14. The composition according to embodiment 12 or embodiment 13, wherein the RNA comprises siRNA.
Embodiment 15. The composition according to embodiment 14, wherein the siRNA targets the human Sparc gene.
Embodiment 16. The composition according to embodiment 14 or embodiment 15, wherein the siRNA comprises a sense strand having at least 80%, 85%, 90%, 95% or 100% sequence identity to the nucleic acid sequence 5′ -AACAAGACCUUCGACUCUUCC-3′.
Embodiment 17. The composition according to any one of embodiments 14 to 16, wherein the siRNA comprises a sense strand having the nucleic acid sequence 5′-AACAAGACCUUCGACUCUUCC-3′.
Embodiment 18. The composition according to any one of embodiments 1 to 17, wherein:
the molar ratio of cations to anionic polymer is between 190: 1 and 260: 1, 200: 1 and 250: 1, 210:1 and 240: 1, or 220: 1 and 230: 1,
the molar ratio of anionic polymer to nucleic acids is between 20: 1 and 90:1, 30: 1 and 80:1, 40: 1 and 70:1, or 50: 1 and 60:1,
the molar ratio of anionic polymer to cations to nucleic acids is between 20 and 100: between 10,000 and 13,000: 1, and/or
the molar ratio of anionic polymer to cations to nucleic acids is about 52: 11,600: 1.
Embodiment 19. The composition according to any one of embodiments 1 to 17, wherein:
the molar ratio of cations to anionic polymer is between 340: 1 and 680: 1, 390: 1 and 620: 1, 430: 1 and 560: 1, or 470: 1 and 480: 1,
the molar ratio of anionic polymer to nucleic acids is between 0.16: 1 and 0.32:1, 0.18: 1 and 0.3:1, 0.19:1 and 0.28:1, or 0.2: 1 and 0.25:1,
the molar ratio of cations to nucleic acids is between 10:1 and 200:1, and/or
the molar ratio of anionic polymer to cations to nucleic acids is about 1.05: 500: 4.6.
Embodiment 20. The composition according to any one of embodiments 1 to 19, wherein the cations are components of a cationic salt included in the composition, and wherein:
the ratio by weight of cationic salt to anionic polymer is between 200:1 and 1:5,
the ratio by weight of anionic polymer to nucleic acids is between 5:1 and 1:4,
the ratio by weight of cationic salt to nucleic acids is between 10:1 and 1:4, and/or
the ratio by weight of anionic polymer to cationic salt to nucleic acids is about 6:6:5.
Embodiment 21. The composition according to any one of embodiments 1 to 20, wherein:
the anionic polymer comprises hyaluronate,
the cations comprise multivalent inorganic cations, and/or
the nucleic acids comprise siRNA,
wherein the hyaluronate has a molecular weight of between 30 and 100 kDa.
Embodiment 22. The composition according to embodiment 21, wherein the multivalent inorganic cations comprise calcium.
Embodiment 23. The composition according to any one of embodiments 1 to 22, wherein the cells are selected from the group consisting of: fibroblasts, endothelial cells, epithelial cells, keratocytes, trabecular meshwork cells, retinal pigment epithelial cells, and any combination thereof.
Embodiment 24. The composition according to any one of embodiments 1 to 23, wherein the cells comprise human cells.
Embodiment 25. The composition according to any one of embodiments 1 to 24, wherein the composition comprises any one or more of:
a solution,
a gel,
nanoparticles,
microparticles,
water-in-oil emulsion,
oil-in-water emulsion,
an implantable polymer, and
foam.
Embodiment 26. The composition according to any one of embodiments 1 to 25, wherein the composition comprises a hydrogel.
Embodiment 27. The composition according to any one of embodiments 1 to 25, wherein the composition comprises nanoparticles.
Embodiment 28. The composition according to any one of embodiments 1 to 27, wherein the composition further comprises a pharmaceutically acceptable excipient or diluent.
Embodiment 29. A method of preparing a composition for the delivery of nucleic acids to cells, the method comprising:
(i) providing an anionic polymer,
(ii) providing cations, wherein the cations do not form part of chitosan or protamine, and
(iii) providing the nucleic acids for delivery to the cells, and
(iv) mixing (i), (ii) and (iii),
   wherein the mixing forms a composition in which the anionic polymer, cations and nucleic acids are bonded by noncovalent interactions.
Embodiment 30. A method of preparing a composition for the delivery of nucleic acids to cells, the method comprising:
(i) providing cations, wherein the cations do not form part of chitosan or protamine,
(ii) providing the nucleic acids for delivery to the cells,
(iii) providing anions,
(iv) mixing (i), (ii) and (iii) to form a mixture,
(v) providing an anionic polymer, and
(vi) mixing the anionic polymer and the mixture,
   wherein the mixing in (vi) forms a composition in which the cations, nucleic acids, anions and anionic polymer are bonded by noncovalent interactions.
Embodiment 31. The method according to embodiment 30, wherein the cations, nucleic acids, and/or anions are mixed with a microemulsion oil phase prior to (iv) and the anionic polymer is mixed with a microemulsion oil phase prior to (vi).
Embodiment 32. The method according to embodiment 31, wherein the mixing with a microemulsion oil phase produces a water-in-oil microemulsion comprising an aqueous phase, wherein the aqueous phase is dispersed as sub-micron droplets.
Embodiment 33. The method according to any one of embodiments 30 to 32, further comprising adding sodium citrate, ethylenediaminetetraacetic acid (EDTA), malate, tartrate, glutamate, histidine, gluconate, lysine, glutamine, methionine, threonine, or any combination thereof.
Embodiment 34. The method according to any one of embodiments 30 to 33, wherein the anions are selected from the group consisting of: monohydrogen phosphate, carbonate, hydrogen carbonate, citrate, sulphate, malate, tartrate, gluconate, aspartate, glutamate, oxalate, malonate, succinate, glutarate, adipate, and any combination thereof.
Embodiment 35. The method according to any one of embodiments 29 to 34, wherein the cations are selected from the group consisting of: multivalent metal ions, calcium, magnesium, manganese, iron, zinc, scandium, titanium, vanadium, chromium, cobalt, nickel, copper, or of the following: glutamine, lysine, arginine, polyglutamine, polylysine, polyarginine, ternary amine-containing compounds, quaternary amine-containing compounds, and any combination thereof.
Embodiment 36. The method according to any one of embodiments 29 to 35, wherein the cations are selected from the group consisting of: calcium, magnesium, polyarginine, and any combination thereof.
Embodiment 37. The method according to any one of embodiments embodiment 29 to 36, wherein the anionic polymer comprises a naturally-occurring anionic polymer.
Embodiment 38. The method according to any one of embodiments 29 to 37, wherein the anionic polymer is selected from the group consisting of: hyaluronate, pectin, cellulose sulphate, alginate, polyacrylic acid, carboxymethyl cellulose, carboxymethyl, dextran, and any combination thereof.
Embodiment 39. The method according to any one of embodiments 29 to 38, wherein the anionic polymer comprises a polymer having a molecular weight of between 30 and 300 kDa, between 35 and 250 kDa, between 40 and 200 kDa, between 45 and 150 kDa, between 50 and 120 kDa, between 60 and 100 kDa, between 50 and 90 kDa, between 70 and 90 kDa or between 30 and 100 kDa.
Embodiment 40. The method according to any one of embodiments 29 to 39, wherein the cations are components of an ionic salt included in the composition.
Embodiment 41. The method according to any one of embodiments 29 to 40, wherein the noncovalent interactions are generated by the cations.
Embodiment 42. The method according to any one of embodiments 29 to 41, wherein the nucleic acids for delivery to cells comprise any one or more of: DNA, RNA and locked nucleic acid (LNA).
Embodiment 43. The method according to embodiment 42, wherein the RNA is selected from the group consisting of: siRNA, miRNA, mRNA, RNA aptamers, ribozymes, circular RNA, and any combination thereof.
Embodiment 44. The method according to embodiment 42 or embodiment 43, wherein the RNA comprises siRNA.
Embodiment 45. The method according to embodiment 44, wherein the siRNA targets the human Sparc gene.
Embodiment 46. The method according to embodiment 44 or embodiment 45, wherein the siRNA comprises a sense strand having at least 80%, 85%, 90%, 95% or 100% sequence identity to the nucleic acid sequence 5′-AACAAGACCUUCGACUCUUCC-3′.
Embodiment 47. The method according to any one of embodiments 44 to 46, wherein the siRNA comprises a sense strand having the nucleic acid sequence 5′-AACAAGACCUUCGACUCUUCC-3′.
Embodiment 48. The method according to any one of embodiments 29 to 47, wherein:
the molar ratio of cations to anionic polymer is between 190: 1 and 260: 1, 200: 1 and 250: 1, 210:1 and 240: 1, or 220: 1 and 230: 1,
the molar ratio of anionic polymer to nucleic acids is between 20: 1 and 90:1, 30: 1 and 80:1, 40: 1 and 70:1, or 50: 1 and 60:1, and/or
the molar ratio of naturally-occurring anionic polymer to cations to nucleic acids is about 52: 11,600: 1.
Embodiment 49. The method according to any one of embodiments 29 to 48, wherein:
the molar ratio of cations to anionic polymer is between 340: 1 and 680: 1, 390: 1 and 620: 1, 430: 1 and 560: 1, or 470: 1 and 480: 1,
the molar ratio of anionic polymer to nucleic acids is between 0.16: 1 and 0.32:1, 0.18: 1 and 0.3:1, 0.19:1 and 0.28:1, or 0.2: 1 and 0.25:1,
the molar ratio of cations to nucleic acids is between 10:1 and 200:1, and/or
the molar ratio of anionic polymer to cations to nucleic acids is about 1.05: 500: 4.6.
Embodiment 50. The method according to any one of embodiments 28 to 49, wherein the cations are components of a cationic salt included in the composition, and wherein:
the ratio by weight of cationic salt to anionic polymer is between 200:1 and 1:5,
the ratio by weight of anionic polymer to nucleic acids is between 5:1 and 1:4,
the ratio by weight of cationic salt to nucleic acids is between 10:1 and 1:4, and/or
the ratio by weight of anionic polymer to cationic salt to nucleic acids is about 6:6:5.
Embodiment 51. The method according to any one of embodiments 29 to 50, wherein:
the anionic polymer comprises hyaluronate,
the cations comprise multivalent inorganic cations, and/or
the nucleic acids comprise siRNA,
wherein the hyaluronate has a molecular weight of between 30 and 100 kDa.
Embodiment 52. The method according to embodiment 51, wherein the multivalent inorganic cations comprise calcium.
Embodiment 53. The method according to any one of embodiments 29 to 52, wherein the cells are selected from the group consisting of: fibroblasts, endothelial cells, epithelial cells, keratocytes, trabecular meshwork cells, retinal pigment epithelial cells, and any combination thereof.
Embodiment 54. The method according to embodiment 53, wherein the cells comprise human cells.
Embodiment 55. The method according to any one of embodiments 29 to 54, wherein the composition comprises any one or more of:
a solution,
a gel,
nanoparticles,
microparticles,
water-in-oil emulsion,
oil-in-water emulsion,
an implantable polymer, and
foam.
Embodiment 56. The method according to any one of embodiments 29 or 35 to 55, wherein the composition comprises a hydrogel.
Embodiment 57. The method according to any one of embodiments 29 to 55, wherein the composition comprises nanoparticles.
Embodiment 58. The method according to any one of embodiments 29 to 57, wherein the composition further comprises a pharmaceutically acceptable excipient or diluent.
Embodiment 59. A composition for the delivery of nucleic acids to cells obtained or obtainable by the method of any one of embodiments 29 to 58.
Embodiment 60. A method of delivering nucleic acids to cells, the method comprising applying the composition of any one of embodiments 1 to 28 or embodiment 59 to the cells.
Embodiment 61. A method of regulating gene expression, the method comprising applying the composition of any one of embodiments 1 to 28 or embodiment 59 to the cells.
Embodiment 62. A method of preventing and/or treating fibrosis in a subject, the method comprising administering to the subject a therapeutically effective amount of the composition of any one of embodiments 1 to 28 or embodiment 59.
Embodiment 63. A method of treating an ocular disease in a subject, the method comprising administering to the subject a therapeutically effective amount of the composition of any one of embodiments 1 to 28 or embodiment 59.
Embodiment 64. Use of the composition of any one of embodiments 1 to 28 or embodiment 59 for the manufacture of a medicament for delivering nucleic acids to cells.
Embodiment 65. Use of the composition of any one of embodiments 1 to 28 or embodiment 59 for the manufacture of a medicament for regulating gene expression.
Embodiment 66. Use of the composition of any one of embodiments 1 to 28 or embodiment 59 for the manufacture of a medicament for the prevention and/or treatment of fibrosis in a subject in need thereof.
Embodiment 67. Use of the composition of any one of embodiments 1 to 28 or embodiment 59 for the manufacture of a medicament for the treatment of an ocular disease in a subject in need thereof.
Embodiment 68. A composition of any one of embodiments 1 to 28 or embodiment 59 for use in delivering nucleic acids to cells.
Embodiment 69. A composition of any one of embodiments 1 to 28 or embodiment 59 for use in regulating gene expression.
Embodiment 70. A composition of any one of embodiments 1 to 28 or embodiment 59 for use in preventing and/or treating fibrosis in a subject.
Embodiment 71. A composition of any one of embodiments 1 to 28 or embodiment 59 for use in treating an ocular disease in a subject.
Embodiment 72. The method of embodiment 60 or the use of embodiment 64 or embodiment 68, wherein the nucleic acids comprise siRNA.
Embodiment 73. The method or the use of embodiment 72, wherein the siRNA targets the human Sparc gene.
Embodiment 74. The method or the use of embodiment 72 or embodiment 73, wherein the siRNA comprises a sense strand having at least 80%, 85%, 90%, 95% or 100% sequence identity to the nucleic acid sequence 5′-AACAAGACCUUCGACUCUUCC-3′.
Embodiment 75. The method of embodiment 61 or the use of embodiment 65 or embodiment 69, wherein the gene comprises or consists of the Sparc gene.
Embodiment 76. The method of embodiment 62 or the use of embodiment 66 or embodiment 70, wherein the fibrosis is subconjunctival fibrosis.
Embodiment 77. The method or the use of embodiment 76, wherein the subconjunctival fibrosis is associated with surgery to treat glaucoma.
Embodiment 78. The method of embodiment 63 or the use of embodiment 67 or embodiment 71, wherein the ocular disease is selected from the group consisting of: glaucoma, retinitis pigmentosa, macular degeneration, diabetic retinopathy and corneal neovascularization.

### Definitions

As used in this application, the singular form “a”, “an” and “the” include plural references unless the context clearly dictates otherwise. For example, the term “cell” also includes multiple cells unless otherwise stated.

As used herein, the term “comprising” means “including”, in a non-exhaustive sense. Variations of the word “comprising”, such as “comprise” and “comprises” have correspondingly varied meanings. Thus, for example, a composition “comprising” a given component A may consist exclusively of component A, or may include one or more additional components such as component B. Similarly, a composition “comprising” an anionic polymer, cations and nucleic acids for delivery to cells may consist exclusively of an anionic polymer, cations and nucleic acids for delivery to cells or may include one or more additional components, for example, water.

As used herein, the term “between” when used in reference to a range of numerical values encompasses the numerical values at each endpoint of the range.

As used herein, the term “about”, when used in reference to a recited numerical value, includes the recited numerical value and numerical values within plus or minus ten percent of the recited value.

As used herein, the term “heteropolymer” means a polymer comprising two or more different types of monomer.

As used herein, the term “multivalent”, when used in reference to an atom and/or element, will be understood to mean an atom and/or element with a valency greater than one.

As used herein, the terms “treat”, “treating”, “treatment”, and the like refer to reducing or ameliorating a disorder/disease and/or symptoms associated therewith. It will be appreciated, although not precluded, that treating a disorder or condition does not require that the disorder, condition, or symptoms associated therewith be completely eliminated.

As used herein, the term “subject” includes any animal of economic, social or research importance including bovine, equine, ovine, primate, avian and rodent species. Hence, a “subject” may be a mammal such as, for example, a human or a non-human mammal.

As used herein, the term “noncovalent”, when used to refer to interactions and/or bonding between atoms and/or molecules, means interactions and/or bonding which do not require the sharing of a pair of electrons. Non-limiting examples of noncovalent interactions and/or bonding include ionic bonds, hydrophobic interactions, hydrogen bonds and Van der Waals forces.

As used herein, the term “siRNA” refers to “small interfering RNA”, also known in the art as “short interfering RNA” and “silencing RNA”. An siRNA is an RNA molecule 20-25 nucleotides in length that is capable of regulating gene expression by degrading the mRNA of a specific target gene as part of the RNA interference pathway.

The terms “hyaluronic acid”, “hyaluronan” and “hyaluronate” may be used interchangeably herein and refer to a linear polyanionic polysaccharide comprised of repeating disaccharide units of glucuronic acid and N-acetyl glucosamine joined by alternating (β-1,3 and β-1,4) glyosidic linkages. “Hyaluronic acid” is also known in the art as “hyaluronan”. “Hyaluronate” is a term commonly used in the art to refer to a salt or ester of “hyaluronic acid”. The “hyaluronic acid”, “hyaluronan” and “hyaluronate” may be naturally-occurring.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, a nucleotide sequence (“subject sequence”) having at least 95% “sequence identity” with another nucleotide sequence (“query sequence”) is intended to mean that the subject sequence is identical to the query sequence except that the subject sequence may include up to five nucleotide alterations per 100 nucleotides of the query sequence. In other words, to obtain a nucleotide sequence of at least 95% sequence identity to a query sequence, up to 5% (i.e. 5 in 100) of the nucleotides in the subject sequence may be inserted or substituted with another nucleotide or deleted.

As used herein, the term “microemulsion” will be understood to mean any liquid mixture having a dispersed phase and a continuous phase, wherein the droplets in the dispersed phase have a diameter of 200 nm or less.

Where reference is made herein to the delivery of agents to cells, it will be understood that the delivery of agents to cells encompasses the delivery of agents to cells and/or into cells. Similarly, where reference is made herein to the delivery of nucleic acids to cells, it will be understood that the delivery of nucleic acids to cells encompasses the delivery of nucleic acids to cells and/or into cells.

### BRIEF DESCRIPTION OF THE FIGURES

Preferred embodiments of the present invention will now be described by way of example only, with reference to the accompanying figures wherein:

FIG. 1 provides graphs depicting the capacity of 5 siRNA delivery formulations to deliver Sparc gene silencing in primary cultured mouse conjunctival fibroblasts. Formulations were tested simultaneously on cells from the same passage as one experiment. mRNA expression of Sparc and Colla1 (where indicated) was measured by real-time quantitative PCR. Values were calculated as folds over those in control cells that were not treated but which were cultured at the same time for the same duration and under the same conditions as the baseline (dotted line).

FIG. 2 provides graphs depicting the results of an evaluation of toxicity. 2000 cells were plated the day before treatment and the cell profile after each treatment was measured over 4 days using the xCELLigence real-time cell analysis (RTCA) assay.

FIG. 3 provides graphs depicting the capacity of a higher concentration of the hyaluronate+calcium (HyA+Ca) formulation for delivery of Sparc gene silencing in primary cultured mouse conjunctival fibroblasts and evaluation of toxicity. Higher concentrations of the formulation were tested simultaneously on cells for both real-time qPCR and cell profile evaluation. FIG. 3a shows mRNA expression of Sparc and Colla1 measured by real-time quantitative PCR. Values were calculated as folds over those in control cells that were not treated but which were cultured at the same time for the same duration and under the same conditions as the baseline (dotted line). FIG. 3b: 2000 cells were plated the day before treatment and the cell profile after each treatment was measured over 4 days using the xCELLigence real-time cell analysis (RTCA) assay.

FIG. 4 provides graphs depicting the capacity of LMW HyA+Ca formulations (second batch) for delivery of Sparc gene silencing in primary cultured mouse conjunctival fibroblasts. mRNA expression of Sparc and Colla1 were measured by real-time quantitative PCR. Values were calculated as folds over those in control cells that were not treated but which were cultured at the same time for the same duration and under the same conditions as the baseline (dotted line). *, p<0.05, Bonferroni-adjusted.

FIG. 5 provides graphs depicting the capacity of two modified formulations for delivery of Sparc gene silencing in primary cultured mouse conjunctival fibroblasts and evaluation of toxicity. Upper panel: formulations 160-03-04, 160-03-05, and 160-03-06; lower panel: formulations 160-03-07, 160-03-08, and 160-03-09. Formulations were tested simultaneously on cells for both real-time qPCR and cell profile evaluation. Left panel, mRNA expression of Sparc and Colla1 were measured by real-time quantitative PCR. Values were calculated as folds over those in control cells that were not treated but which were cultured at the same time for the same duration and under the same conditions as the baseline (dotted line). Right panel, 2000 cells were plated the day before treatment and the cell profile after each treatment was measured over 4 days using the xCELLigence real-time cell analysis (RTCA) assay.

FIG. 6 provides graphs depicting the capacity of freshly-mixed formulations for delivery of Sparc gene silencing in primary cultured mouse conjunctival fibroblasts and evaluation of toxicity. mRNA expression of Sparc and Colla1 were measured by real-time quantitative PCR. Values were calculated as folds over those in control cells that were not treated but which were cultured at the same time for the same duration and under the same conditions as the baseline (dotted line). *, p<0.05 (Bonferroni-adjusted).

FIG. 7 provides graphs depicting the capacity of modified formulations for delivery of Sparc gene silencing in primary cultured mouse conjunctival fibroblasts. Shown in order: formulations 160-07-03, 160-07-06, 160-07-07, 160-07-08, 160-07-10, and 160-07-11. Cells were tested for Sparc mRNA levels by real-time quantitative PCR. Data are normalized relative to Sparc mRNA levels in siScram-treated cells, and relative to 3 different unrelated RNA controls (Actb, 18S, and Rp13a).

FIG. 8 provides graphs depicting the capacity of modified formulations for delivery of Sparc gene silencing in primary cultured mouse conjunctival fibroblasts. The cells were treated with the formulations with poly-arginine at the initial concentration of “1×” (160-07-06) and at 3 times “3×” (160-08-02), with either complete media, or OPTI-MEM media. Cells were tested for real-time qPCR. Data are normalized relative to Sparc mRNA levels in siScram-treated cells, and relative to 3 different unrelated RNA controls (Actb, 18S, and Rp13a).

FIG. 9 is a diagram summarising the method of preparation of the siRNA nanoparticles.

FIG. 10 is a graph which shows the capacity of siRNA nanoparticle formulations for delivery of Sparc gene silencing in primary cultured mouse conjunctival fibroblasts. The cells were treated with the siRNA nanoparticle formulations at three different final concentrations of siRNA in the cell culture medium as shown (2.2 μM, 1.1 μM, and 0.44 μM). Cells were tested for Sparc mRNA levels by real-time qPCR. Data are normalized relative to Sparc mRNA levels in siScram-treated cells, and relative to an unrelated RNA control (Rp13a).

FIG. 11 provides the results of dynamic light scattering (DLS) of the nanoparticles as average size, polydispersity index and histograms, and representative images of the nanoparticles by cryo-TEM.

FIG. 12 provides a graph showing the capacity of siRNA nanoparticle formulations for delivery of Sparc gene silencing in primary cultured mouse conjunctival fibroblasts. The cells were treated with the siRNA nanoparticle formulations at two different final concentrations of siRNA in the cell culture medium as shown (2.2 μM and 4.4 μM). Cells were tested for Sparc mRNA levels by real-time qPCR. Data are normalized relative to Sparc mRNA levels in siScram-treated cells, and relative to an unrelated RNA control (Rp13a).

FIG. 13 provides graphs of Sparc silencing for the calcium-phosphate (CaP)-containing siRNA nanoparticles in mouse conjunctival fibroblasts. Cells were treated with nanoparticles with a final concentration of 2.2 μM (left hand pane) or 4.4 μM (right hand pane) siRNA for three days and analysed for Sparc mRNA by real-time qPCR. Data are expressed relative to the siScram-treated cells, and relative to an unrelated RNA control (Rp13a).

FIG. 14 provides a graph of Sparc silencing for the calcium-phosphate based siRNA nanoparticles in human dermal fibroblasts. Cells were treated with nanoparticles with a final concentration of 2.2 μM siRNA for three days and analysed for Sparc mRNA by real-time qPCR. Data are expressed relative to the siScram-treated cells, and relative to an unrelated RNA control (r18S).

FIG. 15 provides a graph of Sparc silencing for the magnesium-phosphate (middle bars) and calcium-carbonate (right hand bars) siRNA nanoparticles in mouse conjunctival fibroblasts, compared with the calcium phosphate (left hand bars) nanoparticles. Cells were treated with nanoparticles with a final concentration of 2.2 or 4.4 μM siRNA as indicated, for three days and analysed for Sparc mRNA by real-time qPCR. Data are expressed relative to the siScram-treated cells, and relative to an unrelated RNA control (r18S). The CaCO₃ formulation appeared to affect Sparc gene expression at this concentration, resulting in a higher normalized value as shown.

FIG. 16 provides a schematic of the mouse model of conjunctival scarring. The conjunctiva is dissected to reveal the sclera where an incision is made into the anterior chamber. The resulting fistula allows aqueous humour to exit into and underneath the conjunctiva. The accumulated fluid underneath the sutured conjunctiva can be observed as a conjunctival bleb.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### Compositions

The present inventors have developed compositions for the delivery of agents to cells and into cells. The compositions may be used for the delivery of nucleic acids to cells. Without being bound by theory, the present inventors have observed that ionic interactions may allow agents for delivery (e.g. nucleic acids) to become encapsulated. The invention provides compositions suitable for the delivery of agents (e.g. nucleic acids) to biological targets such as tissues and cells. Where reference is made herein to the delivery of agents to cells, it will be understood that the delivery of agents or nucleic acids to cells encompasses the delivery of agents or nucleic acids to cells and/or into cells.

The compositions may comprise an anionic polymer. The anionic polymer may be a naturally-occurring anionic polymer, meaning that it may be formed by natural processes and/or be provided in natural form. In some embodiments of the invention, the anionic polymer comprises or consists of hyaluronic acid, also known in the art as hyaluronan. The anionic polymer may comprise or consist of hyaluronate, which is the ionised form of hyaluronic acid, typically presented as a sodium salt (i.e., sodium hyaluronate).

Hyaluronic acid is a linear polyanionic polysaccharide comprised of repeating disaccharide units of glucuronic acid and N-acetyl glucosamine joined by alternating (β-1,3 and β-1,4) glycosidic linkages. It is a major constituent of the extracellular matrix and may be produced by non-animal sources via fermentation. This ubiquitous anionic polymer is therefore a useful non-limiting example of a polymer for use in the compositions of the invention. Non-limiting examples of other suitable anionic polymers include pectin, cellulose sulphate, alginate, polyacrylic acid, carboxymethyl cellulose, carboxymethyl and dextran.

The compositions described herein may facilitate the delivery of agents into cells via binding of the naturally-occurring anionic polymer to CD44, a ubiquitous transmembrane cell surface molecule. The agents may be nucleic acids. Those skilled in the art would be aware that hyaluronic acid is the major ligand of CD44. In some embodiments of the invention, the cells are fibroblasts. Other suitable cell types may include, but are not limited to, endothelial cells, epithelial cells, keratocytes, trabecular meshwork cells and retinal pigment epithelial cells. The compositions may deliver agents such as nucleic acids to any combination of the aforementioned cell types and/or other cell types. The cells may be from any animal (e.g., a mammal), including, but not limited to, humans, non-human primates, canines, felines, and rodents.

In some embodiments of the invention, the anionic polymer may comprise or consist of alginate, or alginic acid. Alginate is a biocompatible polymer typically obtained from the cell walls of brown seaweed. The properties of alginate are well known to those in the art as it is commonly used in applications such as wound healing, drug delivery, and tissue engineering due to the ease with which it can form a gel.

Anionic polymers used in the present invention may have an average molecular weight of between 30 and 300 kDa, between 35 and 250 kDa, between 40 and 200 kDa, between 45 and 150 kDa, between 50 and 120 kDa, between 60 and 100 kDa, between 50 and 90 kDa, between 70 and 90 kDa or between 30 kDa and 100 kDa. The average molecular weight of the anionic polymer may be, for example, 33 kDa or 78 kDa. The skilled person would easily be able to vary the precise molecular weight of the polymer/s to suit the application.

The compositions may comprise cations. In some embodiments of the invention, the cations do not form part of a cationic heteropolymer. In further embodiments, the cations do not form part of a cationic polymer that is not polymerised amino acids. In still further embodiments, the cations do not form part of chitosan and/or protamine. In certain embodiments, the cations are selected from the group consisting of: multivalent metal ions, glutamine, lysine, arginine, polyglutamine, polylysine, polyarginine, ternary amine-containing compounds, quaternary amine-containing compounds, and any combination thereof. Non-limiting examples of suitable cations include calcium, magnesium and/or polyarginine, which may be poly-L-arginine.

The cations may be multivalent inorganic cations. In some embodiments of the invention, the cations are components of an ionic salt included in the composition. Non-limiting examples of cations that may be used in the compositions include calcium, magnesium, manganese, iron, zinc, scandium, titanium, vanadium, chromium, cobalt, nickel, copper, glutamine, lysine, arginine, polyglutamine, polylysine, polyarginine, ternary amine-containing compounds, quaternary amine-containing compounds, and any combination thereof.

The compositions may comprise anions. Non-limiting examples of suitable anions include phosphate, carbonate, citrate, sulphate, monohydrogen phosphate, hydrogen carbonate, malate, tartrate, gluconate, aspartate, glutamate, oxalate, malonate, succinate, glutarate, adipate, and any combination thereof.

The anionic polymer, cations and agents (e.g. nucleic acids) may be held, i.e. bonded, together in the composition by noncovalent interactions. In some embodiments of the invention, the noncovalent interactions are generated by the cations. Non-limiting examples of noncovalent interactions include ionic bonds, electrostatic interactions, hydrophobic interactions, hydrogen bonds and Van der Waals forces.

The use of naturally-occurring components and/or noncovalent interactions in the compositions may reduce the toxicity of the compositions in comparison to other currently available delivery vehicles. Additionally or alternatively, the naturally-occurring components and/or noncovalent interactions may reduce immunogenicity.

No particular limitation exists in relation to the nucleic acids for delivery to the cells or into the cells. Non-limiting examples of nucleic acids which may be delivered by the compositions of the invention include DNA, RNA, and locked nucleic acid (LNA), and any combination thereof.

The compositions may be suitable for the delivery of therapeutic RNAs. Non-limiting examples of suitable therapeutic RNAs are siRNA, miRNA, mRNA, and RNA aptamers. The compositions may comprise or consist of any combination of RNA classes.

The present inventors have identified optimal molar ratios of cations to anionic polymer, anionic polymer to nucleic acids and anionic polymer to cations to nucleic acids for an exemplary composition of the present invention. These ratios are described in the Examples and claims of the present application. It will be understood that the molar ratios of cations to anionic polymer, anionic polymer to nucleic acids and anionic polymer to cations to nucleic acids disclosed herein are exemplary only. The present inventors have also identified optimal weight ratios of cationic salt to anionic polymer, anionic polymer to nucleic acids, cationic salt to nucleic acids, and anionic polymer to cationic salt to nucleic acids, which will be understood to be exemplary only.

In one exemplary embodiment of the invention, the anionic polymer comprises or consists of hyaluronate with a molecular weight of between 30 and 100 kDa, the cations comprise or consist of multivalent inorganic cations, and/or the nucleic acids comprise or consist of siRNA. In a further embodiment, the anionic polymer comprises or consists of hyaluronate with a molecular weight of between 30 and 100 kDa, the cations comprise or consist of calcium, and/or the nucleic acids comprise or consist of siRNA.

### Methods for Preparing the Compositions

The present invention also provides methods for preparing compositions for the delivery of agents (e.g. nucleic acids) to cells. The methods may comprise providing a solution comprising or consisting of an anionic polymer. In some embodiments, the solution is obtained by dissolving hyaluronic acid in high purity water. The anionic polymer may be hyaluronate. In some embodiments, sodium hyaluronate is dissolved in high purity water. The high purity water used in the preparation of the compositions may be any water substantially free from contaminants. Many types of high purity water are readily available commercially. Additionally or alternatively, the skilled person may prepare high purity water by any one of many well-known methods such as activated carbon, reverse osmosis, ion exchange, filtration and distillation.

The methods may comprise preparing or providing a solution comprising or consisting of cations. In some embodiments, the cations do not form part of a cationic heteropolymer. In some embodiments, the cations do not form part of a cationic polymer that is not polymerised amino acids. In still further embodiments, the cations are not provided as chitosan and/or protamine. The cations may be divalent inorganic cations. The cations may be multivalent cations. In certain embodiments, the cations are selected from the group consisting of: multivalent metal ions, glutamine, lysine, arginine, polyglutamine, polylysine, polyarginine, ternary amine-containing compounds, quaternary amine-containing compounds, and any combination thereof. Non-limiting examples of cations that may be used in the compositions include calcium, magnesium, manganese, iron, zinc, polyglutamine, polylysine, polyarginine, scandium, titanium, vanadium, chromium, cobalt, nickel, copper, glutamine, lysine, arginine, and other organic compounds containing one or more (or a combination of) ternary or quaternary amine groups.

Prior to use in the methods, the cations may be dissolved in high purity water or in a water-miscible pharmaceutically acceptable solvent. The cations may be components of an ionic salt included in the composition, for example, calcium chloride, magnesium chloride or copper chloride. In addition to chloride, other suitable counter ions could include sulphate, phosphate, acetate, citrate, mesylate, nitrate, tartrate, and gluconate. In some embodiments, the cations be components of a water-insoluble salt.

No particular limitation exists in relation to the way in which solutions for use in the methods are prepared. Non-limiting examples of ways in which a solid may be dissolved in high purity water and/or other solvents include heating, swirling, shaking, stirring and vortexing. Persons skilled in the art would be familiar with all of the aforementioned methods.

The methods of the invention may comprise providing the agents (e.g. nucleic acids) to be delivered to the biological target in a solution. The agents (e.g. nucleic acids) may be dissolved in high purity water prior to use in the methods. In some embodiments of the invention, nucleic acids are added to the anionic polymer prior to the addition of cations. The nucleic acids and the anionic polymer may be mixed by stirring, swirling, shaking, etc. Cations may be added to a mixture of the nucleic acids and an anionic polymer and the mixture further mixed by stirring, swirling, shaking, etc. The invention also provides compositions for the delivery of agents (e.g. nucleic acids) to cells produced by the methods of the invention.

Some methods of the invention include providing cations, wherein the cations do not form part of chitosan or protamine, providing the nucleic acids for delivery to the cells, providing anions, mixing the cations, nucleic acids and anions to form a mixture, providing an anionic polymer, and mixing the anionic polymer and the mixture. The cations, nucleic acids, and/or anions may be mixed with a microemulsion oil phase prior to mixing to form the mixture. The anionic polymer may also be mixed with a microemulsion oil phase prior to mixing the anionic polymer and the mixture. In some embodiments, a nucleic acid and anion microemulsion is prepared prior to mixing with the cations. One non-limiting example of a suitable nucleic acid and anion microemulsion is an siRNA and disodium phosphate microemulsion. Mixing with a microemulsion oil phase may produce a water-in-oil microemulsion comprising an aqueous phase. The aqueous phase may dispersed as sub-micron droplets.

The methods of the invention may comprise adding sodium citrate. In some embodiments of the invention, the composition may comprise nanoparticles which may become agglomerated. This problem may be overcome by resuspending the nanoparticles in sodium citrate. This may have the effect of making the composition more suitable for a therapeutic use, for example, injection. In some embodiments, ethylenediaminetetraacetic acid (EDTA), malate, tartrate, glutamate, histidine, gluconate, lysine, glutamine, methionine, threonine, and any combination thereof may be used in addition to or in pace of sodium citrate.

### Exemplary Applications

The present invention also provides methods of delivering agents (e.g. nucleic acids) to cells comprising applying the compositions of the invention to the cells.

The methods may deliver nucleic acids to cells, which may be therapeutic nucleic acids. The wide variety of therapeutic nucleic acids which may be used with the methods of the invention would be well known to those in the art. Non-limiting examples of suitable therapeutic nucleic acids include DNA antisense oligonucleotides, DNA aptamers, locked nucleic acid (LNA), siRNA, miRNA, mRNA, RNA aptamers, ribozymes, circular RNA, and any combination thereof.

Those skilled in the art would be aware of many online tools available to assist the design of therapeutic nucleic acids. For non-limiting examples of online tools suitable for the design of therapeutic small RNAs, see http://rnaidesigner.invitrogen.com/rnaiexpress/design.do, http://www.changbioscience.com/stat/sirna.html and http://wmd3.weigelworld.org/cgi-bin/webapp.cgi.

Without limitation, the compositions of the invention may be useful for the delivery of small interfering RNA (siRNA), also known in the art as short interfering RNA and silencing RNA. An siRNA is an RNA molecule 20-25 nucleotides in length that is capable of regulating gene expression by degrading the mRNA of a specific target gene as part of the RNA interference pathway. Persons skilled in the art are familiar with the enormous therapeutic potential of these small molecules. The invention provides methods of regulating gene expression by applying the compositions of the invention to cells.

By way of non-limiting example, one area where the compositions may find use is in the field of ophthalmology. The main obstacle to achieving long-term surgical success in glaucoma filtration surgery (GFS) is post-operative fibrosis. The inventors of the present invention have previously shown that the Sparc gene can be successfully silenced via the delivery of an siRNA, and that this silencing leads to a reduction in post-GFS scarring. The Sparc gene (secreted protein acidic and rich in cysteine) encodes SPARC, a prototypic calcium binding matricellular protein. Matricellular proteins are secreted glycoproteins that are largely non-structural and involved in mediating cellular interactions with components of the extracellular matrix. SPARC is notably produced at sites of wound healing and tissue remodelling. Collagen is thought to be a key protein regulated by SPARC as well as other extracellular matrix components such as fibronectin and matrix metalloproteinases.

The compositions of the present invention may be used to deliver an siRNA which targets the human Sparc gene to cells and/or into cells. In some embodiments, the siRNA has a sense strand having the nucleic acid sequence 5′-AACAAGACCUUCGACUCUUCC-3′. The siRNA may comprise a sense strand having at least 80%, 85%, 90%, 95% or 100% sequence identity to the nucleic acid sequence 5′-AACAAGACCUUCGACUCUUCC-3′. In one exemplary embodiment of the invention, the naturally-occurring anionic polymer comprises or consists of hyaluronate with a molecular weight of between 50 and 100 kDa, the cations comprise or consist of calcium, and/or the nucleic acids comprise or consist of an siRNA which targets the human Sparc gene and/or has a sense strand having the nucleic acid sequence 5′-AACAAGACCUUCGACUCUUCC-3′. In yet another non-limiting example, the naturally-occurring anionic polymer comprises or consists of hyaluronate with a molecular weight of between 50 and 100 kDa, the cations comprise or consist of calcium, the nucleic acids comprise or consist of an siRNA which targets the human Sparc gene and/or has a sense strand having the nucleic acid sequence 5′-AACAAGACCUUCGACUCUUCC-3′, and/or the molar ratio of cations: naturally-occurring anionic polymer is about 223: 1, the molar ratio of naturally-occurring anionic polymer: nucleic acids is about 52: 1, and/or the molar ratio of hyaluronate: calcium: siRNA is about 52: 11,600: 1.

The present invention provides methods of preventing and/or treating fibrosis in a subject, the methods comprising administering to the subject a therapeutically effective amount of the compositions of the invention. The present invention provides methods of treating ocular diseases in a subject, the methods comprising administering to the subject a therapeutically effective amount of the compositions of the invention. Suitable ocular diseases include any disease of the cornea, conjunctiva and all layers of the retina and optic nerve such as, but not limited to: glaucoma, retinitis pigmentosa, macular degeneration, diabetic retinopathy and corneal neovascularization.

Also provided is the use of the compositions described herein in the manufacture of a medicament for the prevention and/or treatment of fibrosis in a subject and the use of the compositions in the manufacture of a medicament for the treatment of ocular diseases in a subject. The subject may be any animal (e.g., a mammal), including, but not limited to, humans, non-human primates, canines, felines, and rodents. In some embodiments, the fibrosis is subconjunctival fibrosis. In further embodiments, the subconjunctival fibrosis is associated with surgery for glaucoma, for example, glaucoma filtration surgery. The fibrosis may be fibrosis of the skin or of internal organs. The compositions described herein may also be useful for treating fibrosis in wounds and reduction of scarring. Ocular diseases suitable for treatment with the medicaments include any disease of the cornea, conjunctiva and all layers of the retina and optic nerve such as, but not limited to: glaucoma, retinitis pigmentosa, macular degeneration, diabetic retinopathy and corneal neovascularization.

No particular limitation exists in relation to the tissue or organ that the compositions will target for delivery of agents. For example, the compositions may be delivered to the eye, lungs, liver and/or kidney. Using the eye as an example, the compositions could be delivered to the cornea, conjunctiva and/or all the layers of the retina and optic nerve. The compositions may be delivered in the form of, for example, a solution, gel, nanoparticles, microparticles, water-in-oil emulsion, oil-in-water emulsion, implantable polymer, and/or foam.

For therapeutic use, the compositions described herein may be prepared as pharmaceutical compositions containing a therapeutically effective amount of a composition described herein as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. These solutions may be sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and colouring agents, etc. Suitable vehicles and formulations are described, for example, in Remington: The Science and Practice of Pharmacy, 21st Edition, Troy, D. B. ed., Lipincott Williams and Wilkins, Philadelphia, Pa. 2006, Part 5, Pharmaceutical Manufacturing.

No limitation applies in relation to the mode of administration of the compositions. In some embodiments, the compositions are delivered via injection into the subconjunctival space. The mode of administration for therapeutic use of the compositions described herein may be any suitable route that delivers the agents (e.g. nucleic acids) to the subject, such as parenteral administration, e.g., intradermal, intramuscular, intraperitoneal, intravenous and/or subcutaneous; pulmonary; transmucosal; using a formulation in a tablet, capsule, solution, suspension, powder, gel and/or particle; and contained in a syringe, an implanted device, osmotic pump, cartridge and/or micropump; or other means appreciated by the skilled artisan, as well known in the art.

### EXAMPLES

The present invention will now be described with reference to specific Examples, which should not be construed as in any way limiting.

### EXAMPLE ONE

### Determination of the Effectiveness/Efficiency of 5 Prototype Formulations for Delivering Sparc Gene Silencing

To determine the effectiveness/efficiency of 5 formulations for delivering Sparc gene silencing, samples of mouse conjunctival fibroblasts (3×10⁴ cells per 1 ml medium) were each treated in the same fashion with the formulations shown in Table 1 delivering increasing amounts of siRNA, ranging from 0.05 μM to 0.25 μM in 0.05 μM intervals. Sparc mRNA was measured on day 3 following treatment by real-time quantitative PCR. The siRNA used were 21-nucleotide blunt ended RNA duplexes of either a Sparc-specific sequence, 5′-AACAAGACCUUCGACUCUUCC-3′ (referred to as SPARC, or siSPARC), or a scrambled version of this sequence, 5′-GCUCACAGCUCAAUCCUAAUC-3′ (referred to as Scrambled or siScram).

TABLE 1
Formulations and treatment approaches used in Example One

| | Formulation/ | | siRNA/ | siRNA |
|---|---|---|---|---|
| Number | Lot | Formulation | Placebo | Conc |
| on tube | number | approach | Content | (mg/ml) |
| 1 | 160-02-01 | LMW HyA | Placebo | 0 |
| 2 | 160-02-02 | LMW HyA | Scrambled | 0.033 |
| 3 | 160-02-03 | LMW HyA | SPARC | 0.033 |
| 4 | 160-02-04 | HMW HyA | Placebo | 0 |
| 5 | 160-02-05 | HMW HyA | Scrambled | 0.033 |
| 6 | 160-02-06 | HMW HyA | SPARC | 0.033 |
| 7 | 160-02-07 | HyA + Ca | Placebo | 0 |
| 8 | 160-02-08 | HyA + Ca | Scrambled | 0.033 |
| 9 | 160-02-09 | HyA + Ca | SPARC | 0.033 |
| 10 | 160-02-10 | Ca-Alg NP | Placebo | 0 |
| 11 | 160-02-11 | Ca-Alg NP | Scrambled | 0.033 |
| 12 | 160-02-12 | Ca-Alg NP | SPARC | 0.033 |
| 13 | 160-02-13 | Ca-Alg-HyA NP | Placebo | 0 |
| 14 | 160-02-14 | Ca-Alg-HyA NP | Scrambled | 0.033 |
| 15 | 160-02-15 | Ca-Alg-HyA NP | SPARC | 0.033 |

LMW HyA = low molecular weight hyaluronic acid 50-90 kDa;
HMW HyA = ‘high’ molecular weight hyaluronic acid 130-300 kDa;
HyA + Ca = low molecular weight hyaluronic acid complexed with calcium ions;
Ca-Alg NP = calcium alginate nanoparticles;
Ca-Alg-HyA NP = calcium alginate nanoparticles coated with high molecular weight hyaluronic acid.
Note:
0.033 mg/ml siRNA = 2.5 μM.

siRNA Stock Solutions

The siRNA stock solutions used in this Example were prepared using ultrapure water in 2m1 low-DNA binding tubes, mixing the tubes to dissolve the materials in the amounts shown in Tables 2 and 3:

TABLE 2
Scrambled siRNA stock solution

| Material | Amount added | | Final conc. |
|---|---|---|---|
| Scrambled siRNA | 2.9 | mg | (1.4 mg/ml or |
| | | | 104 μM) |
| Ultrapure water | 2.07 | ml | |

TABLE 3
SPARC siRNA stock solution

| Material | Amount added | | Final conc. |
|---|---|---|---|
| SPARC siRNA | 1.9 | mg | (1.4 mg/ml or 104 μM) |
| Ultrapure water | 1.36 | ml | |

HyA and CaCl₂ Stock Solutions

10 mg/ml LMW and HMW HyA, and 8% w/w CaCl₂·2H₂O were made by dissolving the materials in Tables 4, 5 and 6 in ultrapure water in the amounts shown in clean beakers with magnetic stirrers:

TABLE 4
LMW HyA stock solution

| Material | Amount added | Final conc. |
|---|---|---|
| LMW HyA 50-90 kDa | 301.14 mg | 10 mg/ml |
| Ultrapure water | 30.156 g | |

TABLE 5
HMW HyA stock solution

| Material | Amount added | Final conc. |
|---|---|---|
| HMW HyA 130-300 kDa | 206.08 mg | 10 mg/ml |
| Ultrapure water | 20.019 g | |

TABLE 6
CaCl₂ stock solution

| Material | Amount added | Final conc. |
|---|---|---|
| CaCl₂•2H₂O | 322.66 mg | 8.0% w/w |
| Ultrapure water | 4.047 g | |

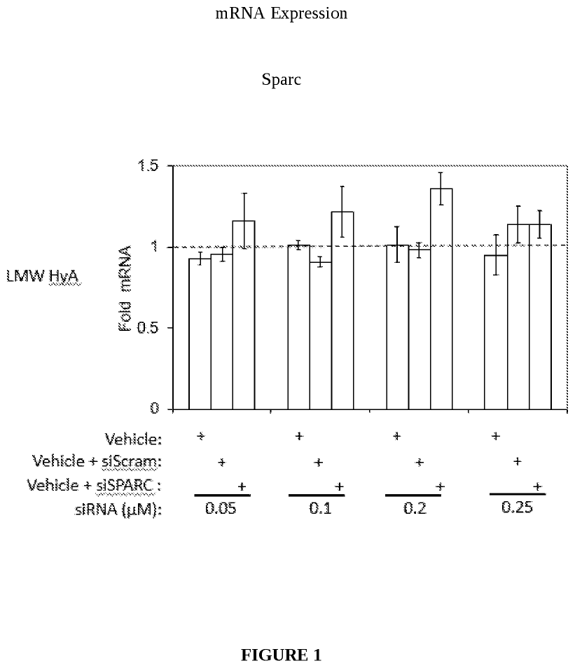

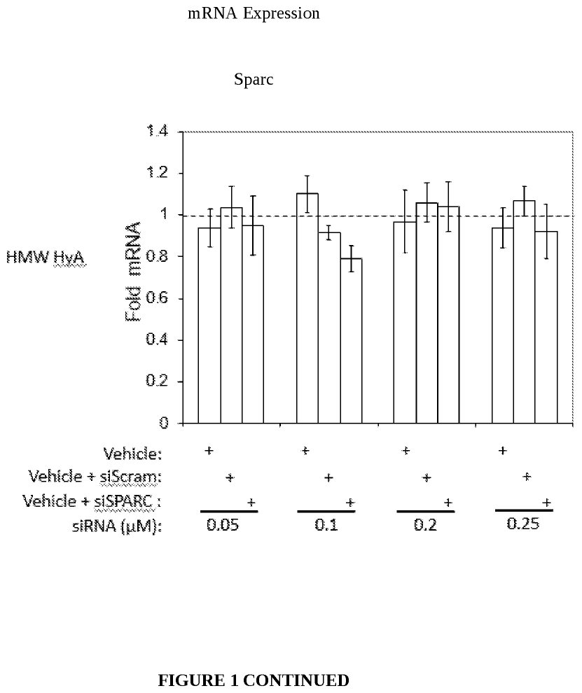

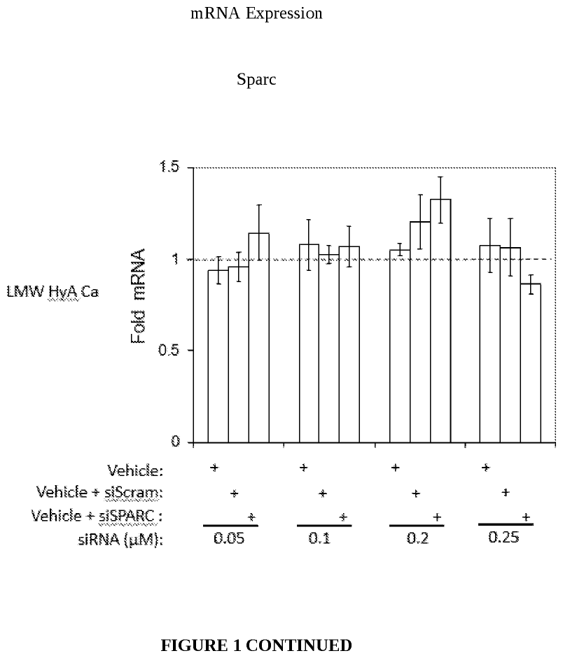

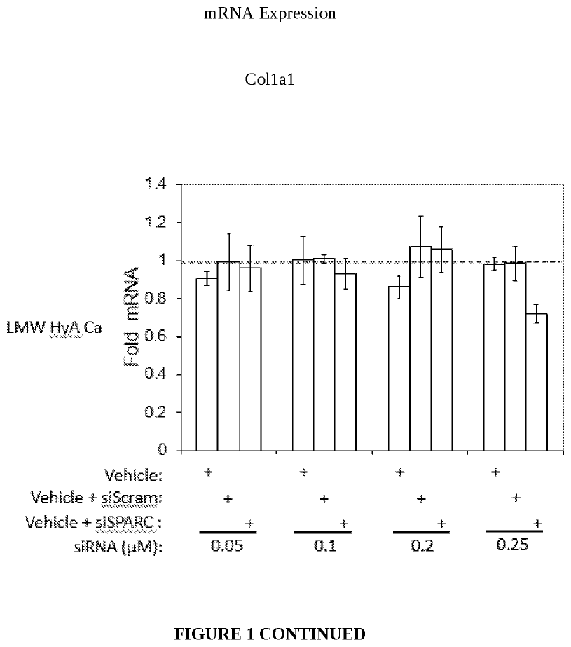

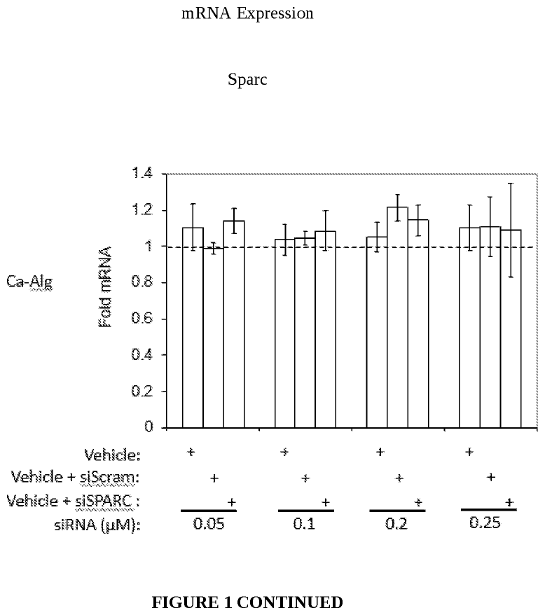

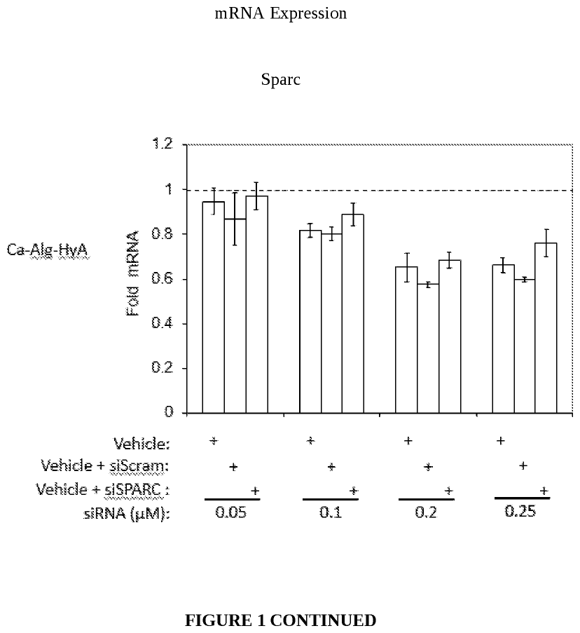

As shown in FIG. 1, HyA+Ca (formulation no. 160-02-09) at 0.25 μM (a ˜1:10 dilution in cell culture medium) resulted in significant downregulation of Sparc mRNA expression by 1.58-fold. This was associated with a reduction in Colla1 mRNA by 1.54-fold.

Preparation of siRNA-HyA-Ca Samples

siRNA-HyA-Ca samples were prepared by stirring the siRNA into the HyA solutions for 20 minutes, then adding the CaCl₂ solution and stirring rapidly until completely mixed. Samples were then filtered through a 0.2 μm syringe filter and aliquoted into sterile 2 ml low-bind tubes (Eppendorf).

Table 7 shows the amounts dispensed in millilitres using electronic pipettes:

TABLE 7
Amounts of materials used in siRNA-HyA-Ca sample preparation

| Sample ID | 160-03-07 | 160-03-08 | 160-03-93 |
|---|---|---|---|
| Material | Placebo | Scrambled | SPARC |
| 10 mg/ml LMW sodium hyaluronate | 7.8 | 3.9 | 3.9 |
| solution | | | |
| Ultrapure water | 0.2 | 0 | 0 |
| Scrambled siRNA stock 1.4 mg/ml | 0 | 0.1 | 0 |
| SPARC siRNA stock 1.4 mg/ml | 0 | 0 | 0.1 |
| 8% (0.6M) Calcium Chloride solution | 0.4 | 0.2 | 0.2 |

The composition of formulations 160-02-07, 160-02-08 and 160-02-09 are provided in Table 8. The composition and molar ratios of formulation 160-02-09 are provided in Table 9.

TABLE 8
Composition of HyA + Ca formulations

| Formulation no. | [siRNA] | [CaCl₂] | [HyA] |
|---|---|---|---|
| 160-02-07 | — | 3.8 mg/ml | 9.3 mg/ml |
| 160-02-08 | 0.033 mg/ml | 3.8 mg/ml | 9.3 mg/ml |
| 160-02-09 | 0.033 mg/ml | 3.8 mg/ml | 9.3 mg/ml |

TABLE 9
Composition of formulation no. 160-02-09

| | | | | Molar | Molar |
|---|---|---|---|---|---|
| | | | | ratio to | ratio to |
| Ingredient | % w/v | mg/ml | Molarity | siRNA | HyA |
| LMW | 0.93 | 9.3 | 130 μM | 52:1 | 1 |
| Sodium | | | | | |
| hyaluronate | | | | | |
| (50-90 kDa) | | | | | |
| SPARC | 0.0033 | 0.033 | 2.5 μM | 1 | 0.019:1 |
| siRNA | | | | | |
| duplex | | | | | |
| Calcium | 0.38 | 3.8 | 29 μM | 11,600:1 | 223:1 |
| Chloride | | | | | |

The molar ratios in formulation no. 160-02-09 were:

Calcium:HyA 223:1

HyA:siRNA 52:1

Formulation no. 160-02-09 did not show inhibition when subsequently tested at 0.5, 0.75 or 1.0 μM, suggesting possible effects of dilution of the cell medium or excess calcium (DMEM cell culture medium contains 1.8 mM calcium).

Although Ca-Alg-HyA NP appeared to result in reduced Sparc mRNA expression, the effect is likely due to non-specific effects rendered by the nanoparticles themselves since both control nanoparticles without siRNA or complexed with si-Scram caused similar suppression of Sparc expression. The other 3 formulations did not appear to inhibit Sparc mRNA expression at the concentrations tested.

To determine that any effect on Sparc mRNA expression was not due to cellular toxicity induced by the formulations, the treated cell profiles were measured over 4 days using the xCELLigence real-time cell analysis (RTCA) assay which detects cell status including cell number, shape/size, and attachment.

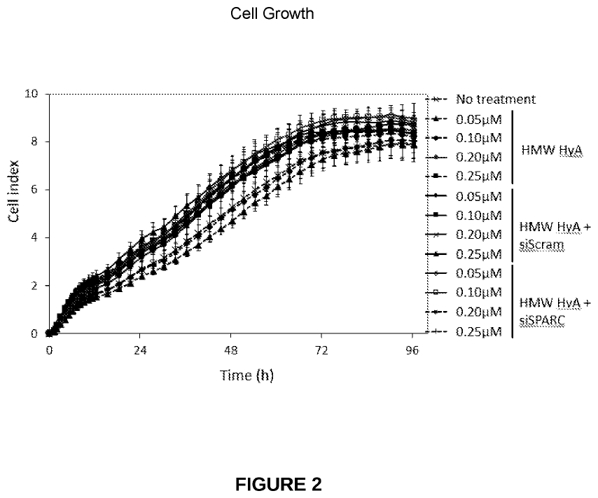

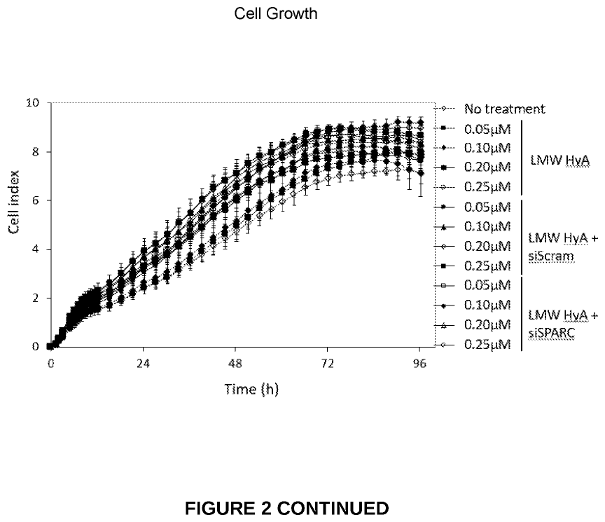

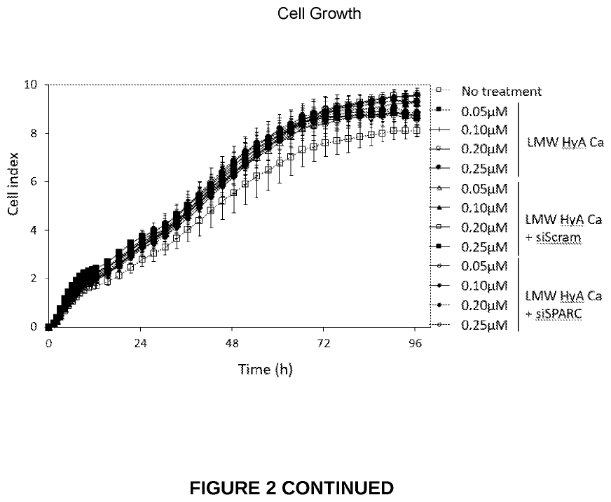

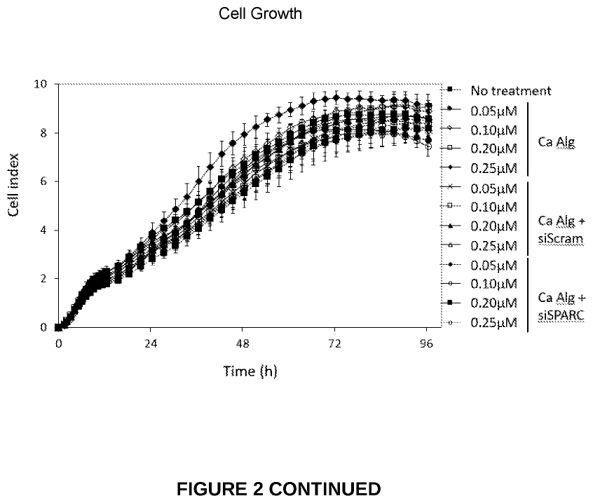

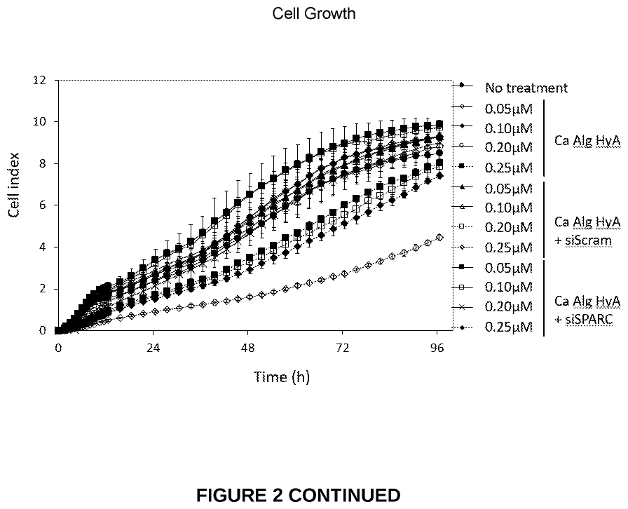

As shown in FIG. 2, only Ca-Alg-HyA NP, both alone or complexed with siRNA, appeared to cause disruption to normal cell growth, particularly when used at the concentration that delivers an equivalent of 0.25 μM siRNA. The potential non-specific toxicity of Ca-Alg-HyA NP may be the cause of Sparc mRNA suppression observed above. The other formulations did not appear to cause measurable alterations to cell profiles at the concentrations tested.

Conclusion

The data from this Example indicate that HyA+Ca at 0.25 μM may be effective in delivering Sparc silencing by at least 1.5-fold.

The experiment in this Example was duplicated and the results were very similar to those set out above.

### EXAMPLE TWO

### Determination of the Effectiveness/Efficiency of Higher Concentrations of the Prototype HyA+Ca Formulation for Selivering Sparc Gene Silencing

A new batch of primary mouse conjunctival fibroblasts was treated with the same batch of HyA+Ca used in Example One, but at higher concentrations.

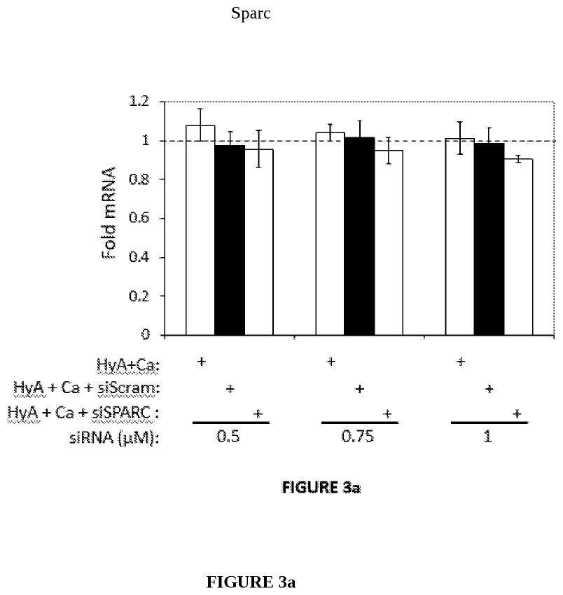

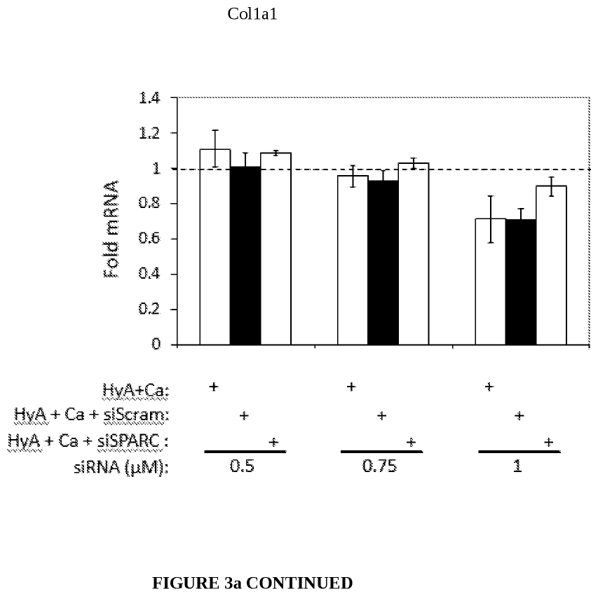

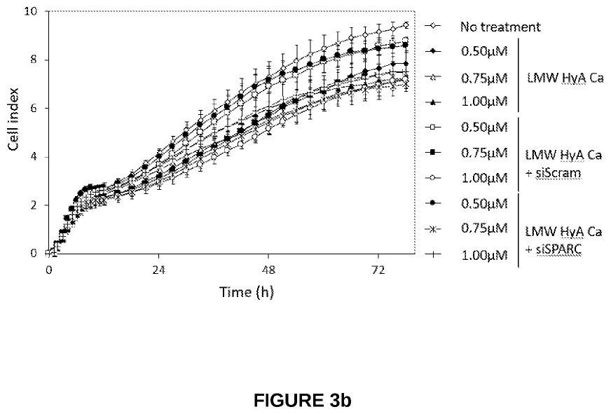

Silencing of Sparc and Colla1 expression was not detected at the higher concentrations of HyA+Ca tested (FIG. 3). Alterations in cell profile may also be observed with increased concentrations of the formulation.

### EXAMPLE THREE

### Determination of the Effectiveness/Efficiency of Two Modified Prototype Formulations for Selivering Sparc Gene Silencing

The aim of this Example is to determine the effectiveness/efficiency of two modified prototype formulations with 5× the amount of siRNA and ⅓ the amount of calcium when compared to the HyA+Ca formulations of the previous two Examples, with either LMW HyA (50-90kDa) or HMW HyA (130-300kDa) for delivering Sparc gene silencing.

TABLE 10
Summary of formulations used in Example Three

| | | | siRNA or/ | |
|---|---|---|---|---|
| Sample | Formulation/Lot | Formulation | Placebo | siRNA |
| Number | number | approach | Content | (mg/ml) |
| 1 | F160-02-07/ | LMW HyA + Ca | Placebo | 0 |
| | L162-03-01 | (repeat of previous | | |
| | | samples) | | |
| 2 | F160-02-08/ | LMW HyA + Ca | Scrambled | 0.035 |
| | L162-03-02 | (repeat of previous | | |
| | | samples) | | |
| 3 | F160-02-09/ | LMW HyA + Ca | SPARC | 0.035 |
| | L162-03-03 | (repeat of previous | | |
| | | samples) | | |
| 4 | F/L160-03-04 | LMW HyA + Ca | Placebo | 0 |
| | | (5× siRNA & | | |
| | | 1/3^{rd} Calcium) | | |
| 5 | F/L160-03-05 | LMW HyA + Ca | Scrambled | 0.175 |
| | | (5× siRNA & | | |
| | | 1/3^{rd} Calcium) | | |
| 6 | F/L160-03-06 | LMW HyA + Ca | SPARC | 0.175 |
| | | (5× siRNA & | | |
| | | 1/3^{rd} Calcium) | | |
| 7 | F/L160-03-07 | HMW HyA + Ca | Placebo | 0 |
| | | (5× siRNA & | | |
| | | 1/3^{rd} Calcium) | | |
| 8 | F/L160-03-08 | HMW HyA + Ca | Scrambled | 0.175 |
| | | (5× siRNA & | | |
| | | 1/3^{rd} Calcium) | | |
| 9 | F/L160-03-09 | HMW HyA + Ca | SPARC | 0.175 |
| | | (5× siRNA & | | |
| | | 1/3^{rd} Calcium) | | |

LMW HyA + Ca = low molecular weight hyaluronic acid 50-90 kDa complexed with calcium ions;
HMW HyA + Ca = ‘high’ molecular weight hyaluronic acid 130-300 kDa complexed with calcium ions.
HyA concentration: 10 mg/ml.
Note:
0.035mg/ml siRNA = 2.6 μM; 0.175 mg/ml siRNA = 13 μM.

Formulation Preparation

Stock solutions of the SPARC or Scrambled duplex siRNA were prepared by dissolving the siRNA in ultrapure water at a concentration of 1.4 mg/mL (104 μM).

Stock solutions of 10 mg/mL LMW (50 to 90 kDa) or HMW (130 to 300 kDa) sodium hyaluronate and 8% w/w calcium chloride dihydrate were prepared by dissolving in ultrapure water.

The siRNA-HyA−Ca samples were then prepared by stifling the siRNA into the HyA solutions for 20 minutes, then adding the required amount of the calcium chloride solution and stirring rapidly until completely mixed. The samples were filtered through 0.2 μm filters and stored at 2 to 8° C. in sterile centrifuge tubes.

Tables 11-13 show the amount (in mL) of the stock solutions used to prepare each formulation.

TABLE 11
Stock solutions of the HyA + Ca formulations
of the previous two Examples
Set 1: repeat of previous LMW HyA + Ca samples

| | Sample ID | | |
|---|---|---|---|
| | L160-03-01 | L160-03-02 | L160-03-03 |
| | Placebo | Scrambled | SPARC |
| Material | (mL) | (mL) | (mL) |
| 10 mg/ml LMW sodium | 7.8 | 3.9 | 3.9 |
| hyaluronate solution | | | |
| Ultrapure water | 0.2 | 0 | 0 |
| Scrambled siRNA stock | 0 | 0.1 | 0 |
| 1.4 mg/ml | | | |
| SPARC siRNA stock | 0 | 0 | 0.1 |
| 1.4 mg/ml | | | |
| 8% (0.6M) Calcium | 0.4 | 0.2 | 0.2 |
| Chloride solution | | | |

TABLE 12
Stock solutions of the LMW HyA + Ca
(5× siRNA and 1/3 calcium) formulations
Set 2: LMW HyA + Ca (5× siRNA and 1/3 calcium)

| | Sample ID | | |
|---|---|---|---|
| | L160-03-04 | L160-03-05 | L160-03-06 |
| | Placebo | Scrambled | SPARC |
| Material | (mL) | (mL) | (mL) |
| 10 mg/ml LMW sodium | 7.8 | 3.9 | 3.9 |
| hyaluronate solution | | | |
| Ultrapure water | 1.0 | 0 | 0 |
| Scrambled siRNA stock | 0 | 0.5 | 0 |
| 1.4 mg/ml | | | |
| SPARC siRNA stock | 0 | 0 | 0.5 |
| 1.4 mg/ml | | | |
| 2.67% Calcium | 0.4 | 0.2 | 0.2 |
| Chloride solution | | | |
| (prepared as a 1:3 | | | |
| dilution from 8% stock) | | | |

TABLE 13
Stock solutions of the HMW HyA + Ca
(5× siRNA and 1/3 calcium) formulations
Set 3: HMW HyA + Ca (5× siRNA and 1/3 calcium)

| | Sample ID | | |
|---|---|---|---|
| | L160-03-07 | L160-03-08 | L160-03-09 |
| | Placebo | Scrambled | SPARC |
| Material | (mL) | (mL) | (mL) |
| 10 mg/ml HMW sodium | 7.8 | 3.9 | 3.9 |
| hyaluronate solution | | | |
| Ultrapure water | 1.0 | 0 | 0 |
| Scrambled siRNA stock | 0 | 0.5 | 0 |
| 1.4 mg/ml | | | |
| SPARC siRNA stock | 0 | 0 | 0.5 |
| 1.4 mg/ml | | | |
| 2.67% Calcium | 0.4 | 0.2 | 0.2 |
| Chloride solution | | | |
| (prepared as a 1:3 | | | |
| dilution from 8% stock) | | | |

Cell Treatment

Mouse conjunctival fibroblasts (3×10⁴ cells per 1 ml medium) were treated with the formulations delivering increasing amounts of siRNA, ranging from 0.10 μM to 1 μM. Sparc mRNA was measured on day 3 after treatment by real-time quantitative PCR.

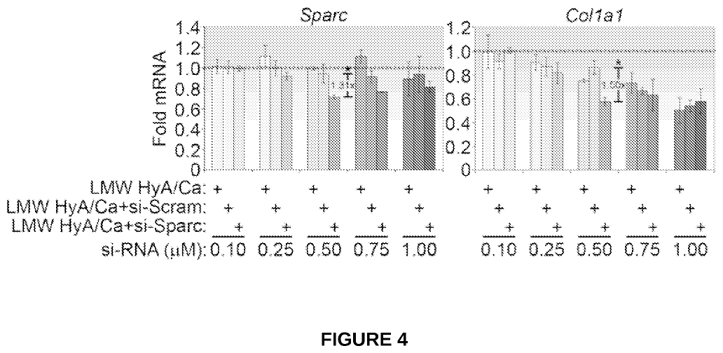

As shown in FIG. 4 (left panel), the repeat experiment for a fresh batch of LMW HyA+Ca formulation demonstrated concentration dependence of the knockdown effect. As examples, treatment with formulations containing 0.25 μM, 0.5 μM and 0.75 μM siSparc can lead to 23%, 40% and 47% Sparc mRNA downregulation from a baseline level of untreated cells respectively. This was associated with a similar trend in Colla1 downregulation. However, it is noted that this delivery formulation can lead to concentration-dependent non-specific downregulation of Colla1 expression.

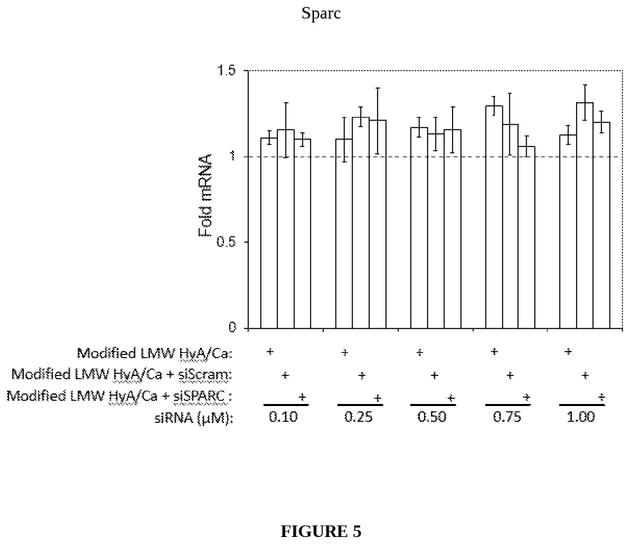

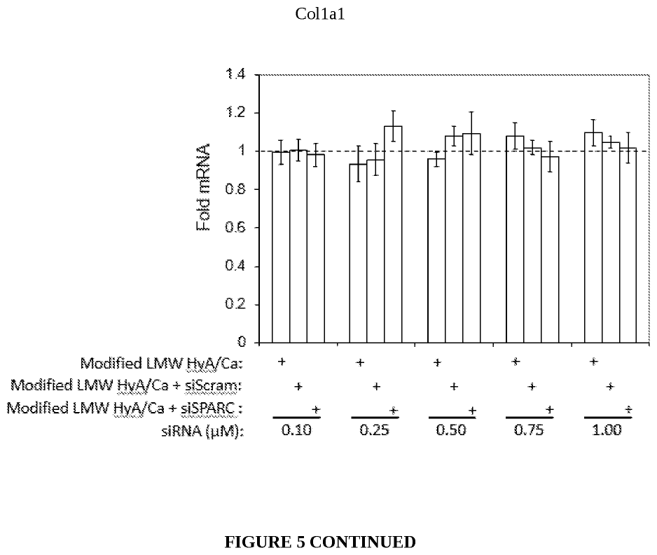

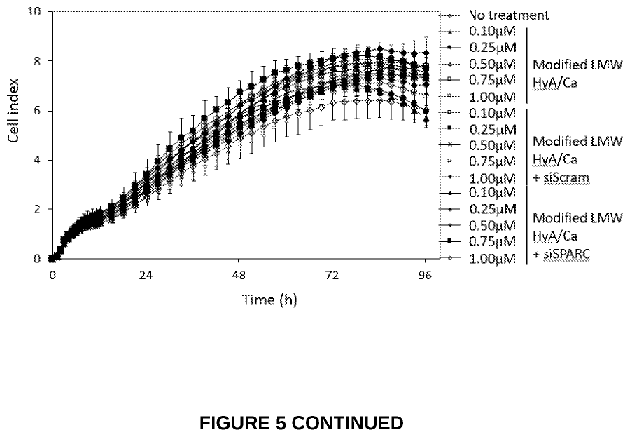

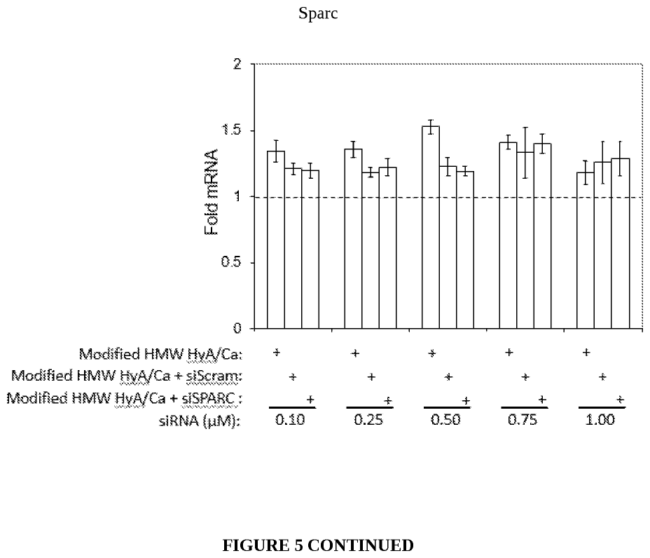

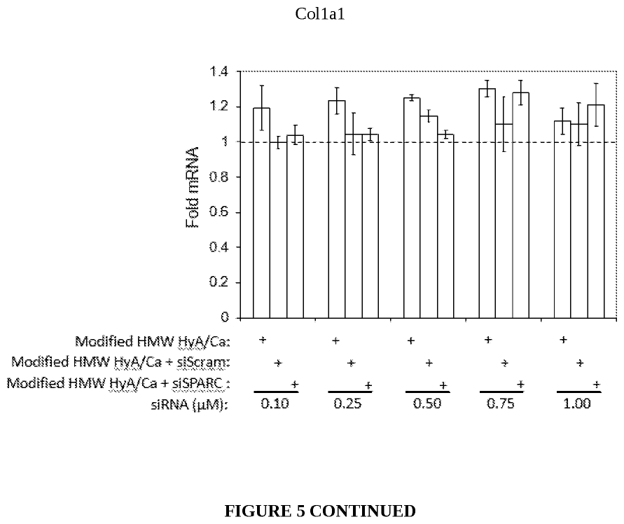

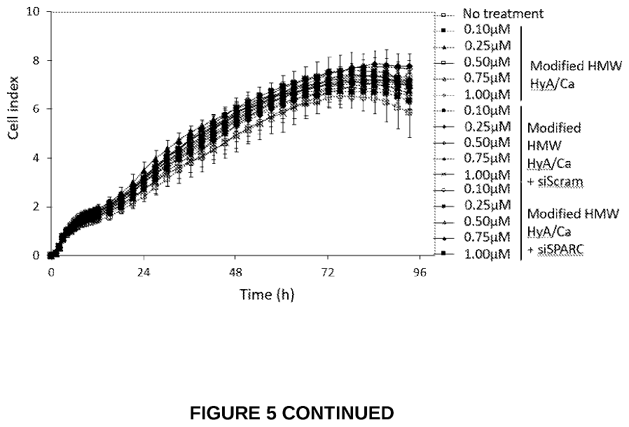

As shown in FIG. 5, neither of the modified formulations resulted in significant downregulation of Sparc mRNA expression at the concentrations tested.

The results in this Example show that LMW HyA+Ca was effective in delivering Sparc silencing and that concentrations of siRNA ranging from 0.25-0.75 μM will be effective.

### EXAMPLE FOUR

### Determination of the Effectiveness of Mixing of Components of Ca, HyA and siRNA just Before Application

Summary of Formulations

The formulations tested in this Example were based on those in previous Examples and are shown in Table 14.

TABLE 14
Forulations tested to determine the effectiveness of mixing
of components of Ca, HyA and siRNA just before application.

| | | | siRNA or/ | |
|---|---|---|---|---|
| Sample | | Formulation | Placebo | siRNA |
| Number | Formulation | approach | Content | (mg/ml) |
| 1 | F160-02-07 | LMW HyA + Ca | Placebo | 0 |
| | | (repeat of previous | | |
| | | samples) | | |
| 2 | F160-02-08 | LMW HyA + Ca | Scrambled | 0.035 |
| | | (repeat of previous | | |
| | | samples) | | |
| 3 | F160-02-09 | LMW HyA + Ca | SPARC | 0.035 |
| | | (repeat of previous | | |
| | | samples) | | |

Formulation Preparation

The formulations were prepared by the same method described in the previous Example.

Cell Treatment

Mouse conjunctival fibroblasts (3×10⁴ cells per 1 ml medium) were treated with 1× and 2× volumes of the freshly mixed formulations. Sparc mRNA was measured on day 3 after treatment by real-time quantitative PCR.

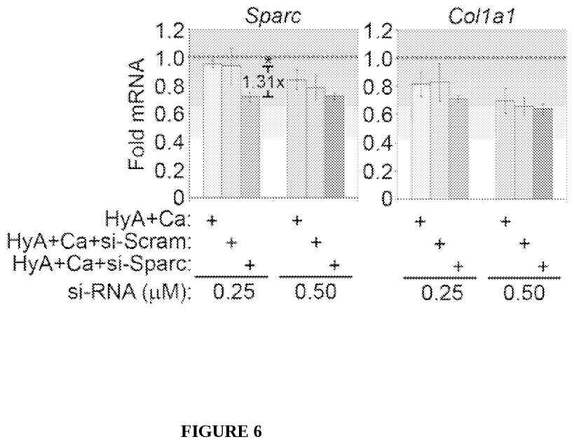

As shown in FIG. 6, the freshly mixed formulation at 0.25 μM siSparc was able to deliver significant Sparc mRNA downregulation when compared with formulation+siS cram. Although downregulation of Sparc mRNA was also observed at 0.5 mM siSparc, this was not significant when compared with formulation+siScram, which, when applied at a higher 2× volume, also appeared to show some effects on Sparc expression.

This Example shows that a freshly mixed LMW HyA+Ca siRNA formulation was effective in delivering Sparc silencing. However, it appears that applying 2× volume of the formulation has effects on gene expression independently of the siRNA.

### EXAMPLE FIVE

### Evaluation of poly-L-arginine in the LMW HyA siRNA Formulation

The aim of this Example was to evaluate the effect of adding another cationic species, poly-L-arginine (5,000 to 15,000 Daltons size range), in place of, or in addition to, the calcium cations in the LMW HyA based siRNA formulation.

Summary of Formulations

An initial set of formulations was prepared as shown in Table 15.

TABLE 15
Initial formulations tested to evaluate the effect of adding poly-L-arginine in
the LMW HyA siRNA formulation.

| | Formulation Description: | | | | | |
|---|---|---|---|---|---|---|
| | | siRNA + | | | | |
| | siRNA + | Hya + | | | | siRNA + |
| | Hya + | poly-Arg + | siRNA + | siRNA + | siRNA + | Ca + poly- |
| | poly-Arg | Ca | Hya | HyA + Ca | poly-Arg | Arg |
| | Formulation ID: | | | | | |
| | 160-07-03 | 160-07-06 | 160-07-07 | 160-07-08 | 160-07-10 | 160-07-11 |
| | μl | μl | μl | μl | μl | μl |
| Sodium hyaluronate (50-90 kDa) | 372 | 372 | 372 | 372 | | |
| 2.5% solution | | | | | | |
| Ultrapure water | 570 | 462 | 580 | 472 | 942 | 834 |
| siRNA (13369.2 Da) 0.14% solution | 48 | 48 | 48 | 48 | 48 | 48 |
| CaCl2•2H2O (147.01 Da) 8% solution | | 108 | 1 | 108 | | 108 |
| Poly-arginine (~10,000) 0.58% solution | 10 | 10 | | | 10 | 10 |
| Total | 1000 | 1000 | 1000 | 1000 | 1000 | 1000 |

An additional set of formulations was prepared as shown in Table 16, to further vary the ratio of poly-L-arginine and siRNA relative to the hyaluronate and calcium.

TABLE 16
Additional set of formulations tested to further vary the ratio of poly-L-arginine
and siRNA relative to the hyaluronate and calcium.

| | Formulation Description: | | | | | |
|---|---|---|---|---|---|---|
| | siRNA + Hya + | | | | 3× siRNA | |
| | poly-Arg + Ca | 2× p Arg | 3× p Arg | 3× siRNA | 3xpArg | ½× pArg |
| | Formulation ID: | | | | | |
| | 160-07-06 | 160-08-01 | 160-08-02 | 160-08-03 | 160-08-04 | 160-08-05 |
| | μl | μl | μl | μl | μl | μl |
| Sodium hyaluronate (50-90 kDa) | 372 | 372 | 372 | 372 | 372 | 372 |
| 2.5% solution | | | | | | |
| Ultrapure water | 462 | 452 | 442 | 366 | 346 | 467 |
| siRNA (13369.2 Da) 0.14% solution | 48 | 48 | 48 | 144 | 144 | 48 |
| CaCI2.2H2O (147.01 Da) 8% solution | 108 | 108 | 108 | 108 | 108 | 108 |
| Poly-arginine (~10,000) 0.58% solution | 10 | 20 | 30 | 10 | 30 | 5 |
| Total | 1000 | 1000 | 1000 | 1000 | 1000 | 1000 |

Formulation Preparation

The formulations were prepared by dissolving the individual components in water to make stock solutions of siRNA (1.4 mg/mL), calcium chloride dihydrate (8%), sodium hyaluronate 50-90 kDa (25 mg/mL), and poly-L-arginine of 5,000 to 15,000 Daltons molecular weight range (0.58%). The stock solutions were then combined by volume according to the tables above, in the following order: the sodium hyaluronate solution was mixed with the ultrapure water, followed by the siRNA solution, then the calcium chloride solution, and finally the poly-L-arginine solution. After each addition, the formulations were shaken in a sealed vial to thoroughly mix. Two formulations from this set were tested on cells: 160-07-06 and 160-08-02.

Cell Treatment

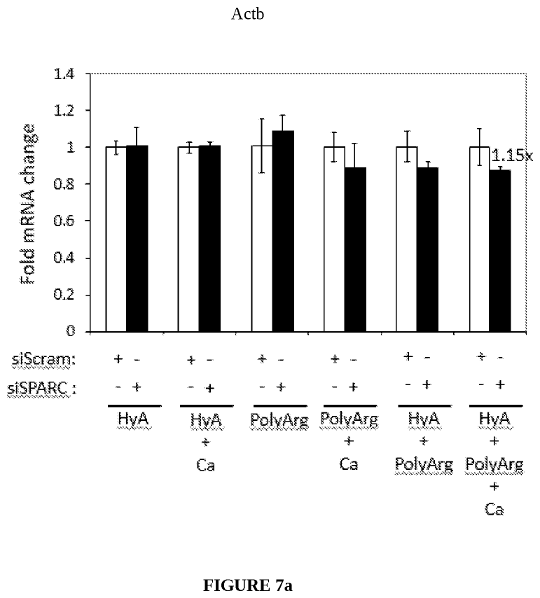

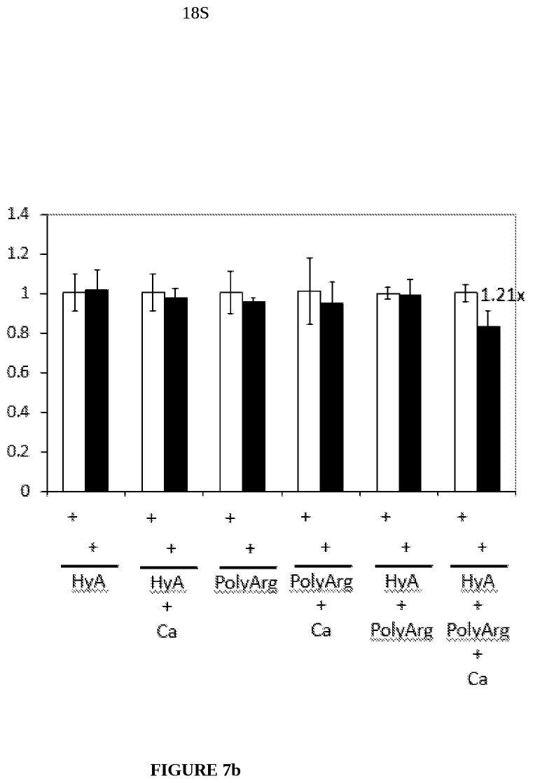

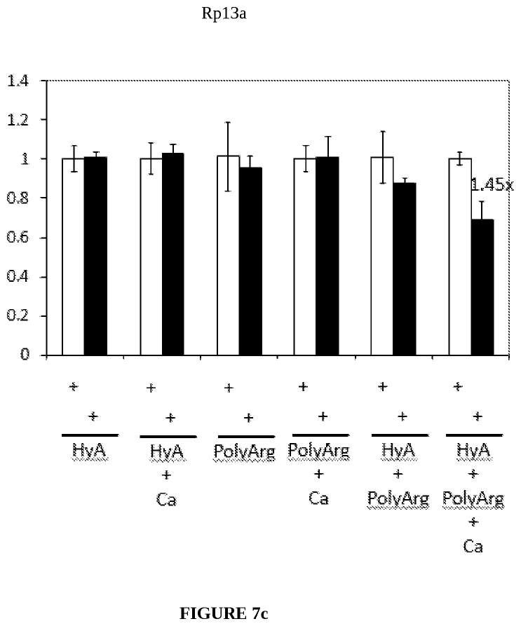

Mouse subconjunctival fibroblasts were treated with the formulations following the same protocol as the previous Examples, then analysed for Sparc silencing by qPCR. Results for the first set of samples tested is shown in FIG. 7.

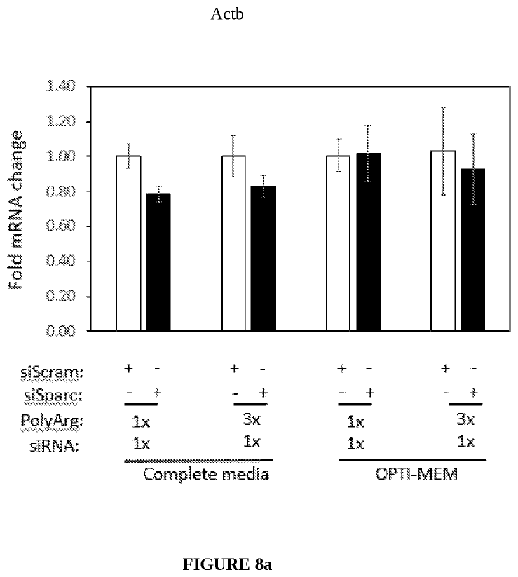

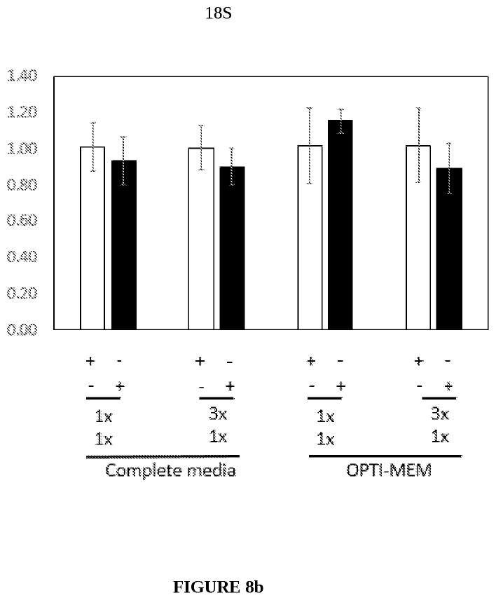

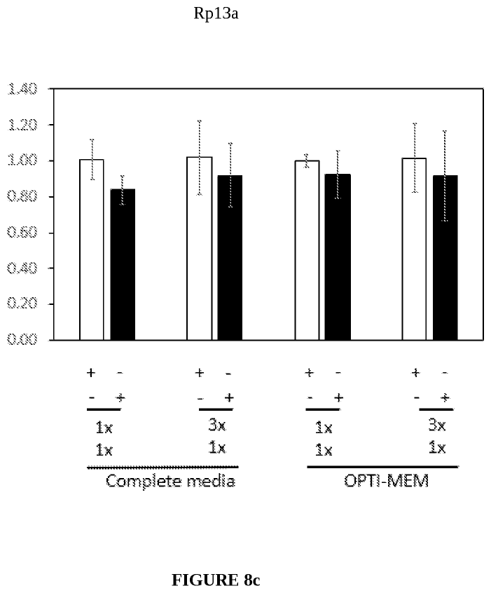

For the second set of samples tested, the Sparc expression data are shown in FIG. 8.

Variable Sparc silencing was observed with the HyA+Ca formulation in this Example. The addition of poly-L-arginine to the formulation in combination with calcium resulted in the greatest Sparc silencing observed. This formulation had the following composition: sodium hyaluronate 50-90 kDa 9.3 mg/mL (approximately 0.13 mM), Calcium Chloride dihydrate 8.64 mg/mL (58.77 mM), siRNA 0.067 mg/mL (5.03 μM), and poly-L-arginine (5-15 kDa) 0.058 mg/mL (5.8 μM).

### EXAMPLE SIX

### Evaluation of a Nanoparticulate Complex of siRNA-HyA-Calcium-phosphate

The formulations of this Example are based on the formulations in the previous Examples, but with the addition of phosphate ions and different ratios of siRNA:HyA:Ca. The formulations were prepared using a water-in-oil microemulsion to control size of the ionic complexes to 200 nm or less. The order of addition was controlled to have a core particle of siRNA-Calcium-Phosphate, with a hyaluronate coating. The use of sodium citrate to resuspend the nanoparticles in a formulation suitable for use was found to prevent the particles from agglomerating.

Summary of Formulations

The formulations are shown in Table 17. The procedure for preparing the siRNA nanoparticle formulations was the same as described in the Example Six.

TABLE 17
Formulations of a nanoparticulate complex of
siRNA-HyA-Calcium-phosphate.

| | | Approximate |
|---|---|---|
| | | siRNA |
| | | content |
| | | when made |
| | | up as |
| Sample ID | Sample Details | 1 mL sample |
| F160-12-14 | Placebo NPs | 0 mg/ml |
| F160-12-15 | SPARC NPs 1 × HyA | 0.6 mg/ml |
| F160-12-16 | Scrambled NPs 1 × HyA | 0.6 mg/ml |
| F160-14-01 | SPARC NPs No HyA | 0.6 mg/ml |
| F160-14-02 | SPARC NPs 0.2 × HyA | 0.6 mg/ml |

Formulation Preparation

The prototype siRNA nanoparticles were prepared by firstly making stock solutions of siRNA, sodium hyaluronate, calcium chloride, and sodium phosphate dissolved in water, then mixing into a microemulsion oil phase to produce water-in-oil microemulsions in which the aqueous phase was dispersed as sub-micron droplets.

The microemulsion oil phase was prepared by mixing Oleth-2, Oleth-10, and Light Mineral Oil at 25:25:50 weight ratio and heating to 40° C.

A siRNA+disodium phosphate microemulsion was prepared by mixing 8.3 parts of a 2% w/w solution of siRNA, 8.3 parts of a 2% w/w disodium phosphate solution, into 75 parts of the microemulsion oil phase, and adding 8.4 parts isopropyl alcohol (by volume), heating to 40° C. and mixing vigorously to form a clear water-in-oil microemulsion.

A calcium chloride microemulsion was prepared by mixing 10 parts of a 2% w/w calcium chloride dihydrate solution in water with 90 parts microemulsion oil phase (by volume), heating to 40° C. and mixing vigorously to form a clear water-in-oil microemulsion.

A sodium hyaluronate microemulsion was prepared by mixing 10 parts of a 0.2% to 1% w/w sodium hyaluronate solution to 90 parts of the microemulsion oil phase and adding 3 parts isopropyl alcohol (by volume) and mixing vigorously to form a clear water-in-oil microemulsion.

The initial calcium-siRNA-phosphate nanoparticles were formed by mixing the siRNA-disodium phosphate microemulsion with the calcium chloride microemulsion at 1:1 volume ratio and storing at 40° C. for 20 minutes.

To incorporate hyaluronate as a secondary layer or coating, the calcium-siRNA-phosphate microemulsion was then mixed with the hyaluronate microemulsion at 50:50 volume ratio, mixed and stored at 40° C. for 20 minutes.

To extract the nanoparticles, the final microemulsion was mixed vigorously with ethanol at 1:1 volume ratio, cooled to 5° C., then centrifuged at 13,400 rpm to collect the precipitated particles and remove the oils and surfactants (discarded with the supernatant). The particles were washed 3 times with ethanol in this way, and then dried to remove the residual ethanol.

Sodium Citrate Buffer

Initial experiments showed that the nanoparticles were agglomerated when suspended in water. It was hypothesized that the use of a buffering and mild chelating agent such as sodium citrate would allow the particles to be more stably suspended in solution. A sample of nanoparticles was suspended in water and then diluted to a final concentration of approximately 0.3 mg/mL in a series of concentrations of trisodium citrate, and analysed for particle size by dynamic light scattering (DLS). As shown in Table 18 below, a 10 mM trisodium citrate buffer resulted in the lowest particle size, approaching the desired size of around 200 nm, which was considered to be efficient for cell uptake by endocytosis.

TABLE 18
Particle size and dynamic light scattering of formulations
with differing concentrations of trisodium citrate.

| | Average | |
|---|---|---|
| | particle | Polydispersity |
| Trisodium citrate | size, | index |
| concentration, mM | nm | (PDI) |
| 500 | 362.7 | 0.202 |
| 250 | 335.3 | 0.343 |
| 100 | 257.9 | 0.205 |
| 50 | 223.7 | 0.249 |
| 10 | 213.8 | 0.232 |
| 0 (water only) | 585.0 | 0.850 |

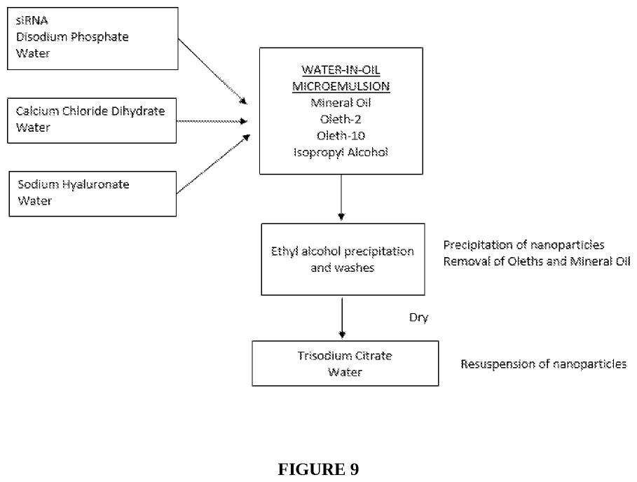

To prepare the final nanoparticles in an aqueous formulation ready for use, the dried nanoparticles were dispersed in a 10 mM trisodium citrate buffer to a final concentration of approximately 0.5 to 1.0 mg/mL siRNA. Further dilutions were prepared in the sodium citrate buffer as required. The diagram in FIG. 9 summarises the method of preparation of the siRNA nanoparticles.

Cell Treatment

Mouse conjunctival fibroblasts (3×10⁴ cells per 1 mL medium) were treated with the formulations delivering increasing amounts of siRNA, ranging from 0.44 μM to 2.2 μM. Sparc mRNA was measured on day 3 after treatment by real-time quantitative PCR.

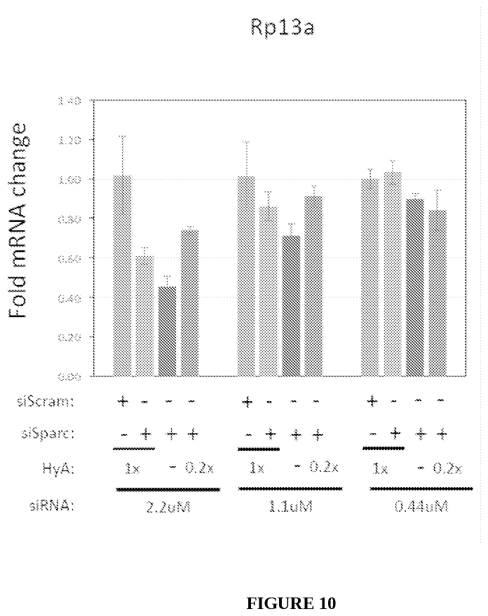

As shown in FIG. 10, the siRNA nanoparticle formulations demonstrated concentration dependence of the knockdown effect.

Significant Sparc gene silencing was observed with the nanoparticles containing SPARC siRNA, in a dose-dependent manner. The formulation with no HyA also showed a significant effect.

### EXAMPLE SEVEN

### Evaluation of Sparc Silencing using a Fresh Sample of the Nanoparticles Tested in Example 6 as Well as Additional Controls Containing the Scrambled siRNA

The aim of this Example was to replicate the findings of Example 6 using freshly prepared siRNA nanoparticle formulations, and to include additional control nanoparticles containing the scrambled siRNA.

Formulation Preparation

Table 19 provides a summary of the formulations used in this Example.

TABLE 19
Formulations used in Example Seven.

| | | Approximate |
|---|---|---|
| | | siRNA |
| | | content |
| | | when made |
| | | up as |
| Sample ID | Sample Details | 1 mL sample |
| F160-15-01 | SPARC NPs No HyA | 0.6 mg/ml |
| | (repeat of 160-14-01) | |
| F160-15-02 | SPARC NPs 0.2 × HyA | 0.6 mg/ml |
| | (repeat of 160-14-02) | |
| F160-15-03 | SPARC NPs 1 × HyA | 0.6 mg/ml |
| | (repeat of 160-12-15) | |
| F160-15-04 | Scrambled NPs No HyA | 0.6 mg/ml |
| F160-15-05 | Scrambled NPs 0.2 × HyA | 0.6 mg/ml |
| F160-15-06 | Scrambled NPs 1 x HyA | 0.6 mg/ml |
| | (repeat of 160-12-16) | |
| F160-15-07 | Placebo NPs 1 × HyA | 0 mg/ml |
| | (repeat of 160-12-14) | |

Size and Structure Analysis by Cryo-Transmission Electron Microscopy (cryo-TEM) and Dynamic Light Scattering (DLS)

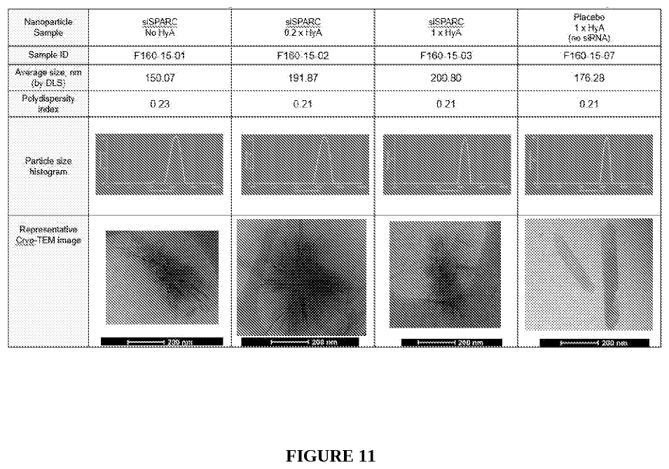

To analyse the particle size, each sample of nanoparticles was dispersed in 10 mM sodium citrate buffer. For dynamic light scattering (DLS), the sample was diluted 1:16 and analysed in an Anton Paar Litesizer instrument, with 90° side scatter and automatic settings. The average size and polydispersity index and histograms are shown in FIG. 11.

The nanoparticles were imaged by cryo-TEM in order to visualize their structure. A humidity-controlled vitrification system was used to prepare the samples for Cryo-TEM. Humidity was kept close to 80% for all experiments, and ambient temperature was 22° C. 300-mesh copper grids coated with perforated carbon film were glow discharged to render them hydrophilic. 3 μl aliquots of the sample were pipetted onto each grid prior to plunging. After 5 seconds adsorption time the grid was blotted manually using Whatman 541 filter paper for approximately 2 seconds. The grid was then plunged into liquid ethane cooled by liquid nitrogen. Frozen grids were stored in liquid nitrogen until required. The samples were examined using a Gatan 626 cryoholder and Tecnai 12 Transmission Electron Microscope at an operating voltage of 120 KV. At all times low dose procedures were followed, using an electron dose of 8-10 electrons/Å2 for all imaging. Images were recorded using a FEI Eagle 4k×4k CCD camera at a range of magnifications using AnalySIS v3.2 camera control software (Olympus). Representative images of the nanoparticles are shown in FIG. 11.

Cell Treatment

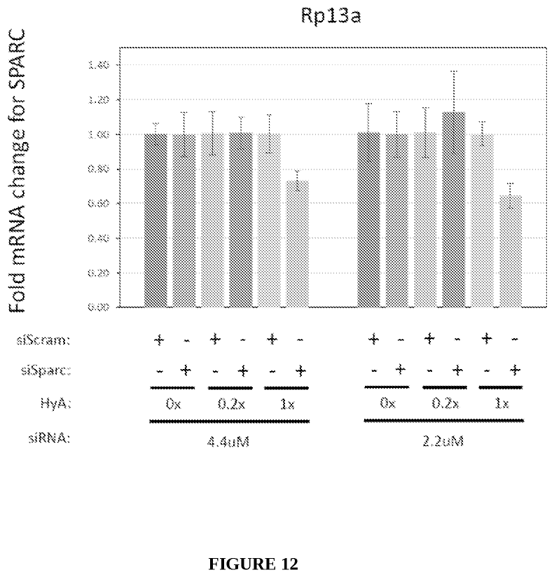

The cell treatment protocol was the same as for Example Six, however in this case, two doses of siRNA were delivered to cells: 2.2 μM and 4.4 μM. As seen in FIG. 12, in this Example, the “1×HyA” formulation showed substantial Sparc silencing, while the other samples including “No HyA” and “0.2×HyA” did not show silencing relative to siScrambled.

Nanoparticles of 150 to 200 nm were produced in this Example with a nano-crystalline structure as visualized by cryo-TEM. In this Example, the ‘1×HyA’ formulation showed significant Sparc silencing, while the ‘0.2×HyA and the ‘No HyA’ samples did not.

### EXAMPLE EIGHT

### Evaluation of the siRNA-HyA-Ca-P Nanoparticles with Varying Average Molecular Weight Hyaluronate (33 kDa, 70 kDa, 78 kDa, 100 kDa), and with Varying Cation and Anion

These formulations were developed to evaluate the effectiveness of the siRNA nanoparticles with 3 different size ranges of pharmaceutical grade sodium hyaluronate (33 kDa, 78 kDa, and 100 kDa average molecular weight), compared with the research grade sodium hyaluronate used in previous experiments (average 70 kDa). In addition, this Example was designed to evaluate variations to the nanoparticle composition, including (i) a different cation (magnesium in place of calcium), and (ii) a different anion (carbonate instead of phosphate).

Formulation Preparation Table 20 provides a summary of the formulations used in this Example.

TABLE 20
Formulations used in Example Eight.

| | | Approximate |
|---|---|---|
| | | siRNA |
| | | content |
| | | when |
| Formulation/ | | made up |
| Lot Number | Sample Details | to 0.5 mL |
| F/L 160-16-01 | siSPARC No HyA | 0.6 mg/mL |
| F/L 160-16-02 | siSPARC 70 kDa HyA | 0.6 mg/mL |
| F/L 160-16-03 | siSPARC 33 kDa HyA | 0.6 mg/mL |
| F/L 160-16-04 | siSPARC 78 kDa HyA | 0.6 mg/mL |
| F/L 160-16-05 | siSPARC 100 kDa HyA | 0.6 mg/mL |
| F/L 160-16-06 | siScrambled No HyA | 0.6 mg/mL |
| F/L 160-16-07 | siScrambled 70 kDa HyA | 0.6 mg/mL |
| F/L 160-16-08 | siScrambled 33 kDa HyA | 0.6 mg/mL |
| F/L 160-16-09 | siScrambled 78 kDa HyA | 0.6 mg/mL |
| F/L 160-16-10 | siScrambled 100 kDa HyA | 0.6 mg/mL |
| F/L 160-16-11 | Placebo 70 kDa HyA | 0 mg/mL |
| F/L 160-17-01 | siSPARC 70 kDa HyA Mg₃(PO₄)₂ | 0.6 mg/mL |
| F/L 160-17-02 | siScrambled 70 kDa HyA Mg₃(PO₄)₂ | 0.6 mg/mL |
| F/L 160-17-03 | Placebo 70 kDa HyA Mg₃(PO₄)₂ | 0 mg/mL |
| F/L 160-17-04 | siSPARC 70 kDa HyA CaCO₃ | 0.6 mg/mL |
| F/L 160-17-05 | siScrambled 70 kDa HyA CaCO₃ | 0.6 mg/mL |
| F/L 160-17-06 | Placebo 70 kDa HyA CaCO₃ | 0 mg/mL |

The protocol for preparing these formulations was the same as in the Example Seven, except, as indicated, different size ranges of sodium hyaluronate were used, or a different cation or anion were used. For the magnesium phosphate samples, magnesium chloride hexahydrate 4.3% w/w water phase was used in place of the calcium chloride water phase, to achieve a 3:2 molar ratio of magnesium:phosphate. For the calcium carbonate samples, sodium bicarbonate 1.14% w/w was used in place of the disodium phosphate to achieve a 1:1 molar ratio of calcium:carbonate.

Cell Treatment

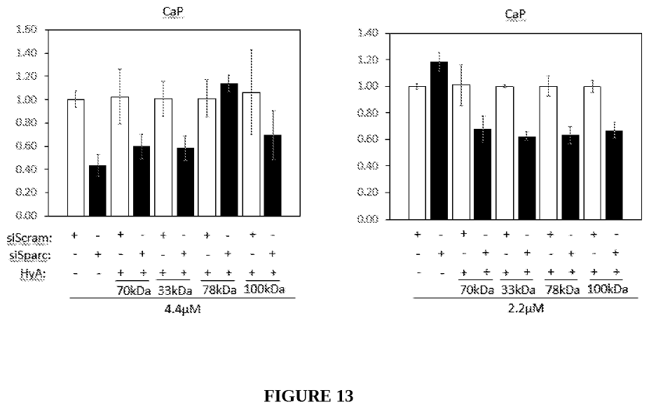

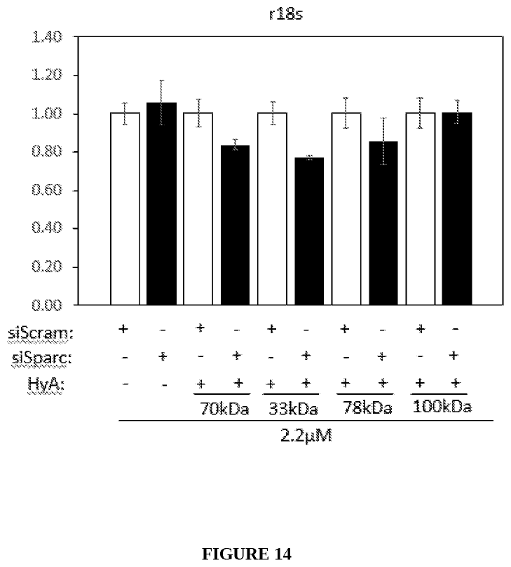

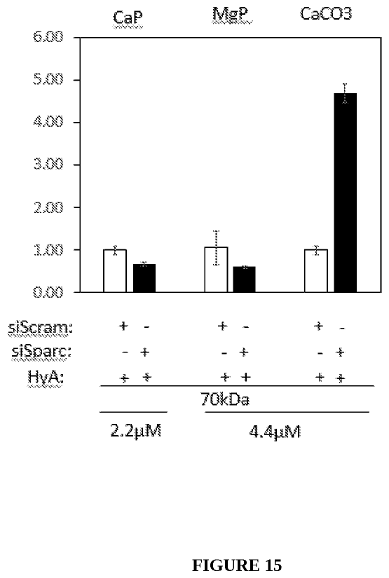

The cell treatment protocol was the same as in Example Seven, however in this case one dose of siRNA was delivered to cells (2.2 μM). Two cell types were evaluated with the formulations: human dermal fibroblasts and mouse subconjunctival fibroblasts. As seen in FIGS. 13-15, in the mouse conjunctival fibroblasts, substantial Sparc silencing was observed for the hyaluronate coated calcium-phosphate-siRNA based particles, while the “No HyA” sample did not show silencing relative to siScramble. In the human dermal fibroblasts, there was a reduction in Sparc expression in the cells treated with samples containing 33 kDa, 70 kDa, and 78 kDa hyaluronate, but not 100 kDa.

The “1×HyA” siRNA nanoparticle formulation containing calcium and phosphate has consistently shown an ability to deliver Sparc gene silencing in fibroblast cells, including mouse conjunctival fibroblasts and human dermal fibroblasts. The 33 kDa, 70 kDa, and 78 kDa hyaluronate appeared to have the most reproduceable effects in both cell types.

### PROPHETIC EXAMPLE NINE

### In Vivo Evaluation of the siRNA-HyA-Ca-P Nanoparticles in Mice

The aim of this Example would be to determine and quantify the gene silencing effect of the siSPARC formulation on suppressing collagen I production and clinical post-op fibrosis in a surgical mouse model of conjunctival fibrosis.

Formulation Preparation

The protocol for preparing the nanoparticles of this Example would be the same as in Examples Six, Seven and Eight, except the final samples would be resuspended using 1/10 of the volume of 100 mM sodium citrate buffer (50 μl per tube) to achieve an siRNA concentration of 450 μM.

In Vivo Evaluation

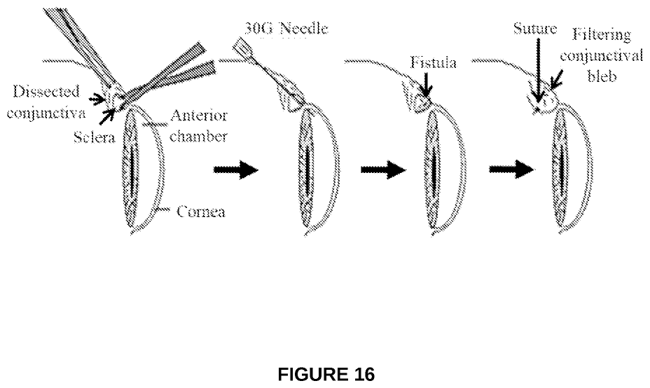

The expression profile of the siSPARC formulation in vivo would be tested in the mouse model of conjunctival scarring as shown in FIG. 16. The mouse model of conjunctival scarring has been validated using MMC. The mouse demonstrated a similar response to humans who have undergone glaucoma surgery when MMC was applied in exactly the same manner.

siRNA-HyA-Ca-P at 2.2 uM (siSPARC) containing 33 kDa and 78 kDa HA would be evaluated in vivo as follows.

5 uL of siSPARC nanoparticles would be injected subconjunctivally at the surgical site at the end of the operation (DO). The animals would be sacrificed on D4 and the eyes harvested for qPCR for expression of Collagen 1 and histological evaluation. Histological visualization of collagen characteristics and bleb morphology collagen architecture in the mouse model of operated conjunctiva would be assessed by hematoxylin and eosin (H&E) staining and picrosirius red staining.

The results of this prophetic Example are expected to illustrate the effect of siSPARC-HyA-Ca-P in reducing collagen 1 gene expression and scar formation as evidenced from qPCR and histology respectively.

1. A composition for the delivery of nucleic acids to cells, the composition comprising:
an anionic polymer,
cations, wherein the cations do not form part of chitosan or protamine, and
the nucleic acids for delivery to the cells,
wherein the anionic polymer, cations and nucleic acids are bonded by noncovalent interactions.

2. The composition according to claim 1, wherein the composition comprises anions.

3. The composition according to claim 1 or claim 2, wherein the anions are selected from the group consisting of: phosphate, monohydrogen phosphate, carbonate, hydrogen carbonate, citrate, sulphate, malate, tartrate, gluconate, aspartate, glutamate, oxalate, malonate, succinate, glutarate, adipate, and any combination thereof.

4. The composition according to any one of claims 1 to 3, wherein the composition comprises sodium citrate, ethylenediaminetetraacetic acid (EDTA), malate, tartrate, glutamate, histidine, gluconate, lysine, glutamine, methionine, threonine, or any combination thereof.

5. The composition according to any one of claims 1 to 4, wherein the cations are selected from the group consisting of: multivalent metal ions, calcium, magnesium, manganese, iron, zinc, scandium, titanium, vanadium, chromium, cobalt, nickel, copper, or of the following: glutamine, lysine, arginine, polyglutamine, polylysine, polyarginine, ternary amine-containing compounds, quaternary amine-containing compounds, and any combination thereof.

6. The composition according to any one of claims 1 to 5, wherein the cations are selected from the group consisting of: calcium, magnesium, polyarginine, and any combination thereof.

7. The composition acording to any one of claims 1 to 6, wherein the anionic polymer comprises a naturally-occurring anionic polymer.

8. The composition according to any one of claims 1 to 7, wherein the anionic polymer is selected from the group consisting of: hyaluronate, pectin, cellulose sulphate, alginate, polyacrylic acid, carboxymethyl cellulose, carboxymethyl, dextran, and any combination thereof.

9. The composition according to any one of claims 1 to 8, wherein the anionic polymer comprises a polymer having a molecular weight of between 30 and 300 kDa, between 35 and 250 kDa, between 40 and 200 kDa, between 45 and 150 kDa, between 50 and 120 kDa, between 60 and 100 kDa, between 50 and 90 kDa, between 70 and 90 kDa or between 30 and 100 kDa.

10. The composition according to any one of claims 1 to 9, wherein the cations are components of an ionic salt included in the composition.

11. The composition according to any one of claims 1 to 10, wherein the noncovalent interactions are generated by the cations.

12. The composition according to any one of claims 1 to 11, wherein the nucleic acids for delivery to cells comprise any one or more of: DNA, RNA and locked nucleic acid (LNA).

13. The composition according to claim 12, wherein the RNA is selected from the group consisting of: siRNA, miRNA, mRNA, RNA aptamers, ribozymes, circular RNA, and any combination thereof.

14. The composition according to claim 12 or claim 13, wherein the RNA comprises siRNA.

15. The composition according to claim 14, wherein the siRNA targets the human Sparc gene.

16. The composition according to claim 14 or claim 15, wherein the siRNA comprises a sense strand having at least 80%, 85%, 90%, 95% or 100% sequence identity to the nucleic acid sequence 5′-AACAAGACCUUCGACUCUUCC-3′.

17. The composition according to any one of claims 14 to 16, wherein the siRNA comprises a sense strand having the nucleic acid sequence 5′-AACAAGACCUUCGACUCUUCC-3′.

18. The composition according to any one of claims 1 to 17, wherein:
the molar ratio of cations to anionic polymer is between 190: 1 and 260: 1, 200: 1 and 250: 1, 210:1 and 240: 1, or 220: 1 and 230: 1,
the molar ratio of anionic polymer to nucleic acids is between 20: 1 and 90:1, 30: 1 and 80:1, 40: 1 and 70:1, or 50: 1 and 60:1,
the molar ratio of anionic polymer to cations to nucleic acids is between 20 and 100: between 10,000 and 13,000: 1, and/or
the molar ratio of anionic polymer to cations to nucleic acids is about 52: 11,600: 1.

19. The composition according to any one of claims 1 to 17, wherein:
the molar ratio of cations to anionic polymer is between 340: 1 and 680: 1, 390: 1 and 620: 1, 430: 1 and 560: 1, or 470: 1 and 480: 1,
the molar ratio of anionic polymer to nucleic acids is between 0.16: 1 and 0.32:1, 0.18: 1 and 0.3:1, 0.19:1 and 0.28:1, or 0.2: 1 and 0.25:1,
the molar ratio of cations to nucleic acids is between 10:1 and 200:1, and/or
the molar ratio of anionic polymer to cations to nucleic acids is about 1.05: 500: 4.6.

20. The composition according to any one of claims 1 to 19, wherein the cations are components of a cationic salt included in the composition, and wherein:
the ratio by weight of cationic salt to anionic polymer is between 200:1 and 1:5,
the ratio by weight of anionic polymer to nucleic acids is between 5:1 and 1:4,
the ratio by weight of cationic salt to nucleic acids is between 10:1 and 1:4, and/or
the ratio by weight of anionic polymer to cationic salt to nucleic acids is about 6:6:5.

21. The composition according to any one of claims 1 to 20, wherein:
the anionic polymer comprises hyaluronate,
the cations comprise multivalent inorganic cations, and/or
the nucleic acids comprise siRNA,
wherein the hyaluronate has a molecular weight of between 30 and 100 kDa.

22. The composition according to claim 21, wherein the multivalent inorganic cations comprise calcium.

23. The composition according to any one of claims 1 to 22, wherein the cells are selected from the group consisting of: fibroblasts, endothelial cells, epithelial cells, keratocytes, trabecular meshwork cells, retinal pigment epithelial cells, and any combination thereof.

24. The composition according to any one of claims 1 to 23, wherein the cells comprise human cells.

25. The composition according to any one of claims 1 to 24, wherein the composition comprises any one or more of:
a solution,
a gel,
nanoparticles,
microparticles,
water-in-oil emulsion,
oil-in-water emulsion,
an implantable polymer, and
foam.

26. The composition according to any one of claims 1 to 25, wherein the composition comprises a hydrogel.

27. The composition according to any one of claims 1 to 25, wherein the composition comprises nanoparticles.

28. The composition according to any one of claims 1 to 27, wherein the composition further comprises a pharmaceutically acceptable excipient or diluent.

29. A method of preparing a composition for the delivery of nucleic acids to cells, the method comprising:
(i) providing an anionic polymer,
(ii) providing cations, wherein the cations do not form part of chitosan or protamine, and
(iii) providing the nucleic acids for delivery to the cells, and
(iv) mixing (i), (ii) and (iii),
wherein the mixing forms a composition in which the anionic polymer, cations and nucleic acids are bonded by noncovalent interactions.

30. A method of preparing a composition for the delivery of nucleic acids to cells, the method comprising:
(i) providing cations, wherein the cations do not form part of chitosan or protamine,
(ii) providing the nucleic acids for delivery to the cells,
(iii) providing anions,
(iv) mixing (i), (ii) and (iii) to form a mixture,
(v) providing an anionic polymer, and
(vi) mixing the anionic polymer and the mixture,
wherein the mixing in (vi) forms a composition in which the cations, nucleic acids, anions and anionic polymer are bonded by noncovalent interactions.

31. The method according to claim 30, wherein the cations, nucleic acids, and/or anions are mixed with a microemulsion oil phase prior to (iv) and the anionic polymer is mixed with a microemulsion oil phase prior to (vi).

32. The method according to claim 31, wherein the mixing with a microemulsion oil phase produces a water-in-oil microemulsion comprising an aqueous phase, wherein the aqueous phase is dispersed as sub-micron droplets.

33. The method according to any one of claims 30 to 32, further comprising adding sodium citrate, ethylenediaminetetraacetic acid (EDTA), malate, tartrate, glutamate, histidine, gluconate, lysine, glutamine, methionine, threonine, or any combination thereof.

34. The method according to any one of claims 30 to 33, wherein the anions are selected from the group consisting of: monohydrogen phosphate, carbonate, hydrogen carbonate, citrate, sulphate, malate, tartrate, gluconate, aspartate, glutamate, oxalate, malonate, succinate, glutarate, adipate, and any combination thereof.

35. The method according to any one of claims 29 to 34, wherein the cations are selected from the group consisting of: multivalent metal ions, calcium, magnesium, manganese, iron, zinc, scandium, titanium, vanadium, chromium, cobalt, nickel, copper, or of the following: glutamine, lysine, arginine, polyglutamine, polylysine, polyarginine, ternary amine-containing compounds, quaternary amine-containing compounds, and any combination thereof.

36. The method according to any one of claims 29 to 35, wherein the cations are selected from the group consisting of: calcium, magnesium, polyarginine, and any combination thereof.

37. The method according to any one of claims claims 29 to 36, wherein the anionic polymer comprises a naturally-occurring anionic polymer.

38. The method according to any one of claims 29 to 37, wherein the anionic polymer is selected from the group consisting of: hyaluronate, pectin, cellulose sulphate, alginate, polyacrylic acid, carboxymethyl cellulose, carboxymethyl, dextran, and any combination thereof.

39. The method according to any one of claims 29 to 38, wherein the anionic polymer comprises a polymer having a molecular weight of between 30 and 300 kDa, between 35 and 250 kDa, between 40 and 200 kDa, between 45 and 150 kDa, between 50 and 120 kDa, between 60 and 100 kDa, between 50 and 90 kDa, between 70 and 90 kDa or between 30 and 100 kDa.

40. The method according to any one of claims 29 to 39, wherein the cations are components of an ionic salt included in the composition.

41. The method according to any one of claims 29 to 40, wherein the noncovalent interactions are generated by the cations.

42. The method according to any one of claims 29 to 41, wherein the nucleic acids for delivery to cells comprise any one or more of: DNA, RNA and locked nucleic acid (LNA).

43. The method according to claim 42, wherein the RNA is selected from the group consisting of: siRNA, miRNA, mRNA, RNA aptamers, ribozymes, circular RNA, and any combination thereof.

44. The method according to claim 41 or claim 42, wherein the RNA comprises siRNA.

45. The method according to claim 44, wherein the siRNA targets the human Sparc gene.

46. The method according to claim 44 or claim 45, wherein the siRNA comprises a sense strand having at least 80%, 85%, 90%, 95% or 100% sequence identity to the nucleic acid sequence 5′-AACAAGACCUUCGACUCUUCC-3′.

47. The method according to any one of claims 44 to 46, wherein the siRNA comprises a sense strand having the nucleic acid sequence 5′-AACAAGACCUUCGACUCUUCC-3′.

48. The method according to any one of claims 29 to 47, wherein:
the molar ratio of cations to anionic polymer is between 190: 1 and 260: 1, 200: 1 and 250: 1, 210:1 and 240: 1, or 220: 1 and 230: 1,
the molar ratio of anionic polymer to nucleic acids is between 20: 1 and 90:1, 30: 1 and 80:1, 40: 1 and 70:1, or 50: 1 and 60:1, and/or
the molar ratio of naturally-occurring anionic polymer to cations to nucleic acids is about 52: 11,600: 1.

49. The method according to any one of claims 29 to 48, wherein:
the molar ratio of cations to anionic polymer is between 340: 1 and 680: 1, 390: 1 and 620: 1, 430: 1 and 560: 1, or 470: 1 and 480: 1,
the molar ratio of anionic polymer to nucleic acids is between 0.16: 1 and 0.32:1, 0.18: 1 and 0.3:1, 0.19:1 and 0.28:1, or 0.2: 1 and 0.25:1,
the molar ratio of cations to nucleic acids is between 10:1 and 200:1, and/or
the molar ratio of anionic polymer to cations to nucleic acids is about 1.05: 500: 4.6.

50. The method according to any one of claims 29 to 49, wherein the cations are components of a cationic salt included in the composition, and wherein:
the ratio by weight of cationic salt to anionic polymer is between 200:1 and 1:5,
the ratio by weight of anionic polymer to nucleic acids is between 5:1 and 1:4,
the ratio by weight of cationic salt to nucleic acids is between 10:1 and 1:4, and/or
the ratio by weight of anionic polymer to cationic salt to nucleic acids is about 6:6:5.

51. The method according to any one of claims 29 to 50, wherein:
the anionic polymer comprises hyaluronate,
the cations comprise multivalent inorganic cations, and/or
the nucleic acids comprise siRNA,
wherein the hyaluronate has a molecular weight of between 30 and 100 kDa.

52. The method according to claim 51, wherein the multivalent inorganic cations comprise calcium.

53. The method according to any one of claims 29 to 52, wherein the cells are selected from the group consisting of: fibroblasts, endothelial cells, epithelial cells, keratocytes, trabecular meshwork cells, retinal pigment epithelial cells, and any combination thereof.

54. The method according to claim 53, wherein the cells comprise human cells.

55. The method according to any one of claims 29 to 54, wherein the composition comprises any one or more of:
a solution,
a gel,
nanoparticles,
microparticles,
water-in-oil emulsion,
oil-in-water emulsion,
an implantable polymer, and
foam.

56. The method according to any one of claims 29 or 35 to 55, wherein the composition comprises a hydrogel.

57. The method according to any one of claims 29 to 55, wherein the composition comprises nanoparticles.

58. The method according to any one of claims 29 to 57, wherein the composition further comprises a pharmaceutically acceptable excipient or diluent.

59. A composition for the delivery of nucleic acids to cells obtained or obtainable by the method of any one of claims 29 to 58.

60. A method of delivering nucleic acids to cells, the method comprising applying the composition of any one of claims 1 to 28 or claim 59 to the cells.

61. A method of regulating gene expression, the method comprising applying the composition of any one of claims 1 to 28 or claim 59 to the cells.

62. A method of preventing and/or treating fibrosis in a subject, the method comprising administering to the subject a therapeutically effective amount of the composition of any one of claims 1 to 28 or claim 59.

63. A method of treating an ocular disease in a subject, the method comprising administering to the subject a therapeutically effective amount of the composition of any one of claims 1 to 28 or claim 59.

64. Use of the composition of any one of claims 1 to 28 or claim 59 for the manufacture of a medicament for delivering nucleic acids to cells.

65. Use of the composition of any one of claims 1 to 28 or claim 59 for the manufacture of a medicament for regulating gene expression.

66. Use of the composition of any one of claims 1 to 28 or claim 59 for the manufacture of a medicament for the prevention and/or treatment of fibrosis in a subject in need thereof.

67. Use of the composition of any one of claims 1 to 28 or claim 59 for the manufacture of a medicament for the treatment of an ocular disease in a subject in need thereof.

68. A composition of any one of claims 1 to 28 or claim 59 for use in delivering nucleic acids to cells.

69. A composition of any one of claims 1 to 28 or claim 59 for use in regulating gene expression.

70. A composition of any one of claims 1 to 28 or claim 59 for use in preventing and/or treating fibrosis in a subject.

71. A composition of any one of claims 1 to 28 or claim 59 for use in treating an ocular disease in a subject.

72. The method of claim 60 or the use of claim 64 or claim 68, wherein the nucleic acids comprise siRNA.

73. The method or the use of claim 72, wherein the siRNA targets the human Sparc gene.

74. The method or the use of claim 72 or claim 73, wherein the siRNA comprises a sense strand having at least 80%, 85%, 90%, 95% or 100% sequence identity to the nucleic acid sequence 5′-AACAAGACCUUCGACUCUUCC-3′.

75. The method of claim 61 or the use of claim 65 or claim 69, wherein the gene comprises or consists of the Sparc gene.

76. The method of claim 62 or the use of claim 66 or claim 70, wherein the fibrosis is subconjunctival fibrosis.

77. The method or the use of claim 76, wherein the subconjunctival fibrosis is associated with surgery to treat glaucoma.

78. The method of claim 63 or the use of claim 67 or claim 71, wherein the ocular disease is selected from the group consisting of: glaucoma, retinitis pigmentosa, macular degeneration, diabetic retinopathy and corneal neovascularization.
